(19) United States
(12) Patent Application Publication
(10) Pub. No.: US 2004/0260199 A1
(43) Pub. Date: Dec. 23, 2004

(54) Cytology collection device

(75) Inventors: David M. Hardia JR., Winston-Salem, NC (US); John Mugan, Galway (IE); Charles J. Lightdale, Leonia, NJ (US); Courtney Leigh Rawlings, Winston-Salem, NC (US)

Correspondence Address: Brinks Hofer Gilson & Lione, P.O. Box 10395, Chicago, IL, 60610, US

(73) Assignees: Wilson-Cook Medical, Inc.; Cook Ireland Limited

(21) Appl. No.: 10/731,500

(22) Filed: Dec. 9, 2003

Related U.S. Application Data
(63) Continuation-in-part of application No. 10/699,487, filed on Oct. 31, 2003.
(60) Provisional application No. 60/479,709, filed on Jun. 19, 2003.

(51) Int. Cl. A61B 10/00

(52) U.S. Cl. 600/566; 600/569

(57) ABSTRACT

Differing cytology device apparatuses, and methods for their use, are provided for collecting cytology samples from within a mammalian body. The apparatuses and methods disclosed are minimally invasive. Some of the apparatuses include a needle having an inner lumen extending from a proximal to a distal end. A stylet, having a proximal end and a distal end, is adapted to be inserted into the inner lumen of the needle to plug the inner lumen while a cytology sample is cut in order to prevent contamination within the needle. The distal end of the stylet may be sharp, and may be adapted to extend beyond the distal end of the needle to cut a cytology sample. Also included is a cytology collection device which is adapted to be inserted into the inner lumen of the needle when the stylet is withdrawn from the inner lumen of the needle. The distal end of the cytology collection device is extended beyond the distal end of the needle in order to collect the cytology sample, and then retracted inside the needle after collecting the sample. In one embodiment, a cytology collection device comprises an elongate member having a plurality of bristles near its distal end. In another embodiment, a cytology collection device comprises an inflatable balloon. In yet another embodiment, a cytology collection device comprises a wire mesh device. In a further embodiment, the apparatus may include an endoscope which contains a transducer emitting ultrasound waves which are used to determine a position of the cytology collection device within the body.

### REFERENCE TO PREVIOUS APPLICATIONS

This application claims the benefit of U.S. provisional application Ser. No. 60/479,709, filed on Jun. 19, 2003. This application is also a continuation in part of U.S. application Ser. No. 10/699,487, filed on Oct. 31, 2003, titled HANDLE FOR MEDICAL DEVICES, AND MEDICAL DEVICE ASSEMBLIES INCLUDING A HANDLE.

### FIELD OF THE INVENTION

The invention relates generally to the field of medical devices. More specifically, the invention relates to cytology devices for collecting cytology samples from a mammalian body, and to methods of collecting these samples.

### BACKGROUND OF THE INVENTION

The development of minimally invasive methods and devices over recent years has revolutionized the practice of medicine. These methods and devices allow clinicians to perform a wide variety of procedures while minimizing trauma to the patient. Along these lines, there is a need for cytology collection devices and methods for their use which employ minimally invasive technologies in order to collect cytology samples from a mammalian body.

Many of the existing cytology devices suffer from one or more problems. For instance, some of the prior art experiences problems with contamination of the cytology device due to the usage of an open needle lumen to collect the cytology sample. This results from the inadvertent collection of undesirable substances as the open needle lumen traverses through the body during the procedure. Other prior art experiences difficulties in collecting an appropriate sample. This may result from the usage of a small diameter needle to collect the sample without the aid of a cytology collection device such as a brush, balloon, or wire mesh device. Still other prior art experiences difficulty in navigating the cytology device to the precise location within the body. This often occurs due to the lack of a guidance system to indicate where the cytology collection device is located within the body.

It would be beneficial to provide minimally invasive cytology devices and methods for their use which limit one or more of these or other problems.

### BRIEF SUMMARY OF THE INVENTION

The present invention provides cytology devices and methods for their use which employ minimally invasive technologies in order to collect cytology samples from a mammalian body.

In one embodiment, a medical apparatus comprises a needle having a proximal end, a distal end, and an inner lumen extending from the proximal end to the distal end. Also included is a stylet having a proximal end and a distal end. The stylet is adapted to be inserted into and withdrawn from the inner lumen of the needle with at least a portion of the stylet adapted to plug the inner lumen of the needle when a cytology sample is cut. Further included is a cytology collection device having a proximal end and a distal end. The cytology collection device is adapted to be inserted into the inner lumen of the needle when the stylet is withdrawn from the inner lumen of the needle. The distal end of the cytology collection device is adapted to extend beyond the distal end of the needle in order to collect the cytology sample.

In another embodiment, a medical apparatus for collecting a cytology sample from a mammalian body comprises an endoscope containing a transducer. The endoscope defines a working lumen. The apparatus also includes a member having a proximal end, a distal end, and an inner lumen, wherein the member extends into the working lumen of the endoscope. Further included is a cytology collection device having a proximal end, and a distal end for cytology collection. The cytology collection device is adapted to be inserted into the inner lumen of the member, while the distal end of the cytology collection device is adapted to extend beyond the distal end of the member in order to collect the cytology sample. The transducer is adapted to emit ultrasound waves to determine a position of the cytology collection device within the mammalian body using the ultrasound waves.

In yet another embodiment, a method for collecting a cytology sample from a mammalian body is disclosed. An apparatus is provided comprising a needle having a proximal end, a distal end, and an inner lumen extending from the proximal end to the distal end. A stylet is included having a proximal end and a distal end. Also included is a cytology collection device having a proximal end and a distal end for cytology collection. The stylet is inserted into the inner lumen of the needle so that at least a portion of the stylet plugs the inner lumen of the needle. An area within the mammalian body is cut and the stylet is withdrawn from the inner lumen of the needle. The cytology collection device is inserted into the inner lumen of the needle so that the distal end of the cytology collection device extends beyond the distal end of the needle. Finally, the cytology sample is collected from the mammalian body using the cytology collection device, and the distal end of the cytology collection device is retracted into the inner lumen of the needle.

In another embodiment, a method for collecting a cytology sample from a mammalian body is again disclosed. An apparatus is provided comprising an endoscope, wherein the endoscope defines a working lumen and contains a transducer. The apparatus includes a member having a proximal end, a distal end, and an inner lumen, wherein the member extends into the working lumen of the endoscope. Also included is a cytology collection device having a proximal end, and a distal end for cytology collection, wherein the cytology collection device extends into the inner lumen of the member. The working lumen of the endoscope is inserted into the mammalian body, and the distal end of the cytology collection device is extended beyond the distal end of the member. Ultrasound waves are emitted from the transducer of the endoscope, reflected off the cytology collection device, and received using the transducer of the endoscope. A position of the cytology collection device within the mammalian body is then determined.

### BRIEF DESCRIPTION OF SEVERAL VIEWS OF THE DRAWINGS

FIG. 1 is a perspective view of an apparatus for collecting a cytology sample from within a mammalian body according to one embodiment of the invention in which a stylet is used to cut a cytology sample.

FIG. 1A is a magnified view of a portion of the apparatus illustrated in FIG. 1.

FIG. 2 is a perspective view of the apparatus illustrated in FIG. 1 shown in an open configuration.

FIG. 3 is a longitudinal sectional view of the apparatus illustrated in FIG. 1.

FIG. 3A is a magnified view of a portion of the apparatus illustrated in FIG. 3.

FIG. 3B is a magnified view of a portion of the apparatus illustrated in FIG. 3.

FIG. 4 is a longitudinal sectional view of the apparatus illustrated in FIG. 2.

FIG. 4A is a magnified view of a portion of the apparatus illustrated in FIG. 4.

FIG. 4B is a magnified view of a portion of the apparatus illustrated in FIG. 4.

FIG. 5 is a perspective view of an apparatus according to another embodiment of the invention.

FIG. 6 is a sectional view of the apparatus illustrated in FIG. 5 taken along line 6-6.

FIG. 7 is a perspective view of an apparatus according to another embodiment of the invention.

FIG. 7A is a sectional view of the apparatus illustrated in FIG. 7 taken along long line 7A-7A.

FIG. 8 is a perspective view of an apparatus for collecting a cytology sample from within a mammalian body according to one embodiment of the invention in which a cytology brush is used to collect the sample cut by the apparatus of FIG. 1.

FIG. 8A is a magnified view of a portion of the apparatus illustrated in FIG. 8.

FIG. 9 is a perspective view of the apparatus illustrated in FIG. 8 shown in an open configuration.

FIG. 10 is a longitudinal sectional view of the apparatus illustrated in FIG. 8.

FIG. 10A is a magnified view of a portion of the apparatus illustrated in FIG. 10.

FIG. 10B is a magnified view of a portion of the apparatus illustrated in FIG. 10.

FIG. 11 is a longitudinal sectional view of the apparatus illustrated in FIG. 9.

FIG. 11A is a magnified view of a portion of the apparatus illustrated in FIG. 11.

FIG. 11B is a magnified view of a portion of the apparatus illustrated in FIG. 11.

FIG. 12 is a perspective view of an apparatus for collecting a cytology sample from within a mammalian body according to one embodiment of the invention using a cytology balloon to collect the sample.

FIG. 12A is a magnified view of a portion of the apparatus illustrated in FIG. 12.

FIG. 12B is a front view of the balloon assembly, in a deflated position, which forms a portion of the apparatus illustrated in FIG. 12.

FIG. 12C is a front view of the balloon assembly, in an inflated position, which forms a portion of the apparatus illustrated in FIG. 12.

FIG. 13 is a perspective view of an apparatus according to another embodiment of the invention.

FIG. 14 is a perspective view of an apparatus during one step of a method for collecting a cytology sample according to one embodiment of the invention.

FIG. 14A is a perspective view of the apparatus of FIG. 14 during another step of the method.

FIG. 14B is a perspective view of the apparatus of FIG. 14A, substituting a cytology brush for the stylet, during another step of the method.

FIG. 14C is a perspective view of the apparatus of FIG. 14B during another step of the method.

FIG. 14D is a perspective view of the apparatus of FIG. 14C during another step of the method.

FIG. 15 is a flow-chart setting forth a method for collecting a cytology sample according to another embodiment of the invention.

### DETAILED DESCRIPTION OF EXEMPLARY EMBODIMENTS OF THE INVENTION

The following provides a detailed description of several embodiments of the invention. The embodiments described and illustrated herein are exemplary in nature, and are not intended to limit the scope of the invention in any matter. Rather, the description of these embodiments serves simply to aid in enabling one of ordinary skill in the art to make and use the invention.

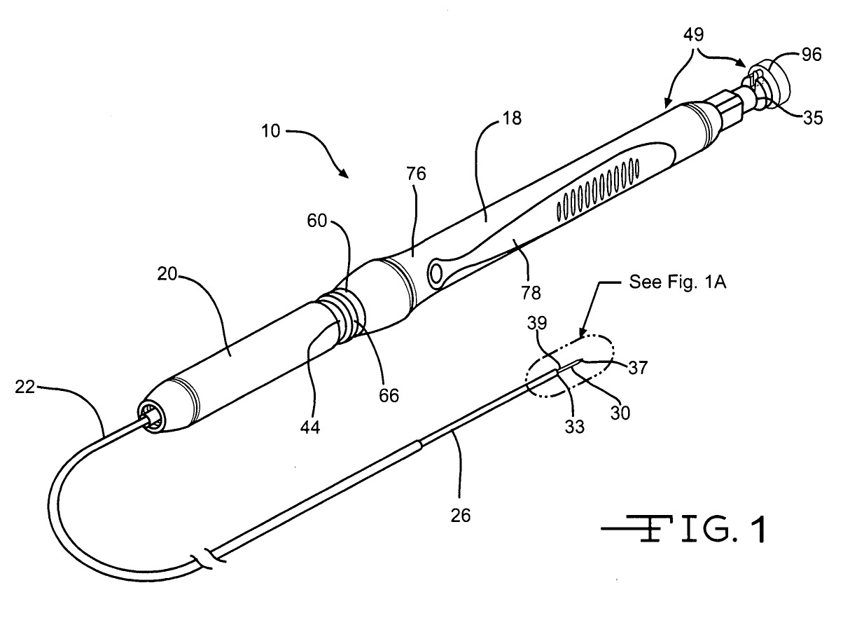

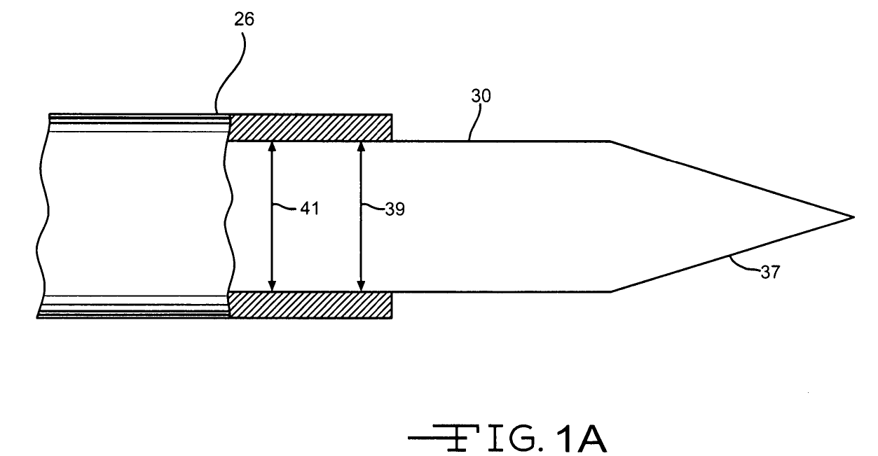

FIGS. 1, 1A, 2-3, 3A, 3B, 4, 4A, and 4B illustrate an apparatus 10 for collecting a cytology sample from within a mammalian body according to one embodiment of the invention. In this embodiment, the apparatus 10 comprises an inner handle member 12 having proximal 14 and distal 16 ends. A first outer handle member 18 is slideably disposed on the proximal end 14 of the inner handle member 12. A second outer handle member 20 is slideably disposed on the distal end 16 of the inner handle member. An elongate sheath 22 is attached to the inner handle member 12 and extends axially beyond the distal end 16 of the inner handle member 12. As used herein, the term “axially” refers to one member situated around, in the direction of, on, or along an axis of another member, and is not limited to one member situated around, in the direction of, on, or along a central axis of another member. The sheath 22 defines a sheath lumen 24.

A needle 26 is attached to the first outer handle member 18 and is at least partially disposed in the sheath lumen 24. In other embodiments, instead of a needle, a first member may be used having a generally tubular ending, or other non-sharp ending. The needle 26 may be adapted to provide suction. The needle 26 may be made of a variety of materials including steel and other types of metals. The needle 26 defines a needle lumen 28 and has a proximal end 31 and a distal end 33. A stylet 30 having a proximal end 35 and a sharp distal end 37 is slideably disposed in the needle lumen 28, and at different times in the procedure is adapted to be completely outside of the needle lumen 28, partially inside the needle lumen 28, or entirely disposed within the needle lumen 28. In other embodiments, the distal end of the stylet may be dull and in a variety of configurations such as rounded. The stylet 30 comprises a solid rod. When the stylet 30 is inserted into the needle lumen 28, the outer circumference 39 of the stylet 30 is flush with the inner diameter 41 of the needle lumen 28. The sharp distal end 37 of the stylet 30 is adapted to extend beyond the needle's distal end 33 by a distance in the range of one centimeter in order to cut an area within the mammalian body so that a cytology sample may be cut by moving the stylet 30 axially. In other embodiments, the distal end 37 of the stylet 30 may extend varying distances from the needle's distal end 30. In further embodiments, the distal end 37 of the stylet 30 is not adapted to extend beyond the needle's distal end 33 in order to cut tissue, but rather its sole purpose is to plug the needle lumen 28 during a cutting procedure. In these embodiments, the needle 26 or other apparatus and/or methods known in the art may be utilized to cut an area within the mammalian body.

A significant advantage of the present invention is that during the cutting of a cytology sample, undesired liquids or substances do not contaminate the needle lumen 28 at the needle's distal end 33. This is due to the stylet 30 plugging the needle's lumen 28 as a result of the outer circumference 39 of the stylet 30 being flush with the inner diameter 41 of the needle lumen 28. The stylet 30 may be made of one or more of steel, metal, and nitinol. In other embodiments, the stylet may be varying shapes including rectangular and polygonal, and may be made of varying materials. The stylet 30 extends axially beyond the second outer handle member 20, and into the sheath lumen 24. As discussed below, the handle 49 is adapted to provide axial movement of the stylet 30 and needle 26. In addition to the preferred embodiment as described, the handle 49 may comprise a variety of types including a finger-loop handle, a pin-vice handle, and a half-finger loop handle.

FIG. 1 illustrates the apparatus 10 according to this embodiment of the invention in a closed configuration. That is, the first 18 and second 20 outer handle members are advanced fully onto their respective portions of the inner handle member 12. In this configuration of this embodiment, the first 18 and second 20 outer handle members envelop the inner handle member 12.

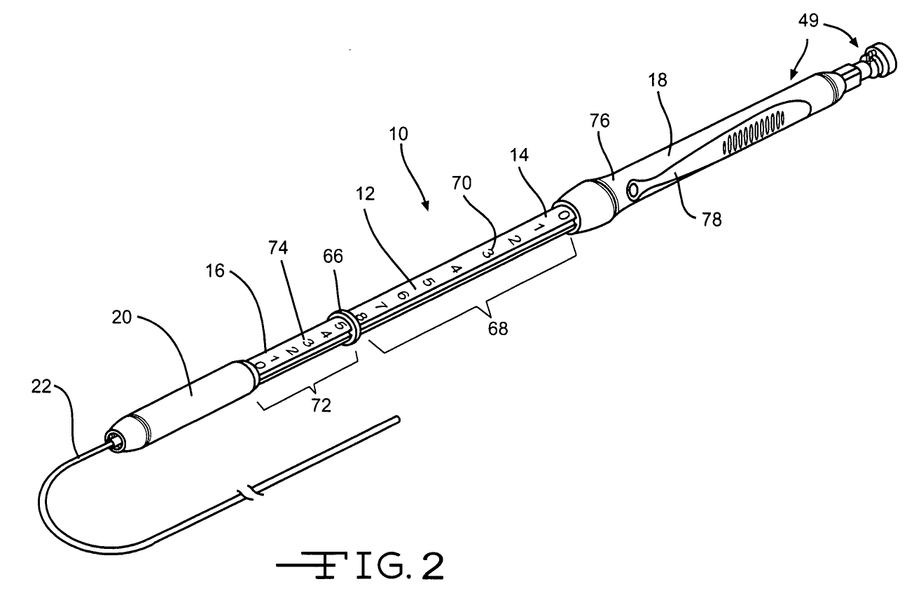

FIG. 2 illustrates the apparatus 10 in an open configuration. In this configuration, both the first 18 and second 20 outer handle members are retracted from their respective positions relative to the inner handle member illustrated in FIG. 1. This open configuration reveals the inner handle member 12. Changing the apparatus 10 from the closed configuration illustrated in FIG. 1 to the open configuration illustrated in FIG. 2 results in different relative positions of various components. For example, because the sheath 22 is attached to the inner handle member 12, movement of the second outer handle member 20 along the inner handle member 12 changes the length by which the sheath 22 extends axially beyond the distal end of the second outer handle member 20. Also, because the needle 26 is attached to the first outer handle member 18, movement of the first outer handle member 18 along the inner handle member 12 changes the position of the needle 26 relative to the sheath 22. Some of this movement may change a length by which the needle 26 extends axially beyond a distal end of the sheath 22. A comparison of FIGS. 1 and 2 illustrates that the needle 26 may extend axially beyond a distal end of the sheath 22 when the apparatus 10 is in a closed configuration (FIG. 1), but may be completely within the sheath 22 when the handle is in an open configuration (FIG. 2).

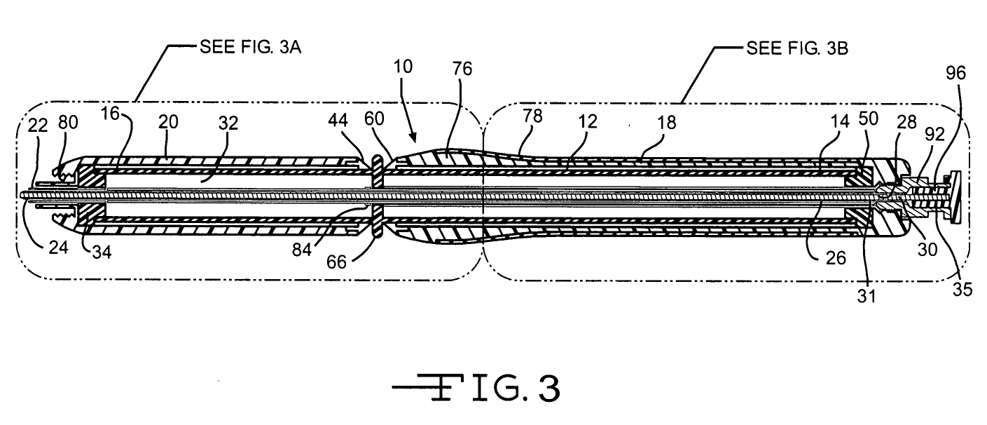

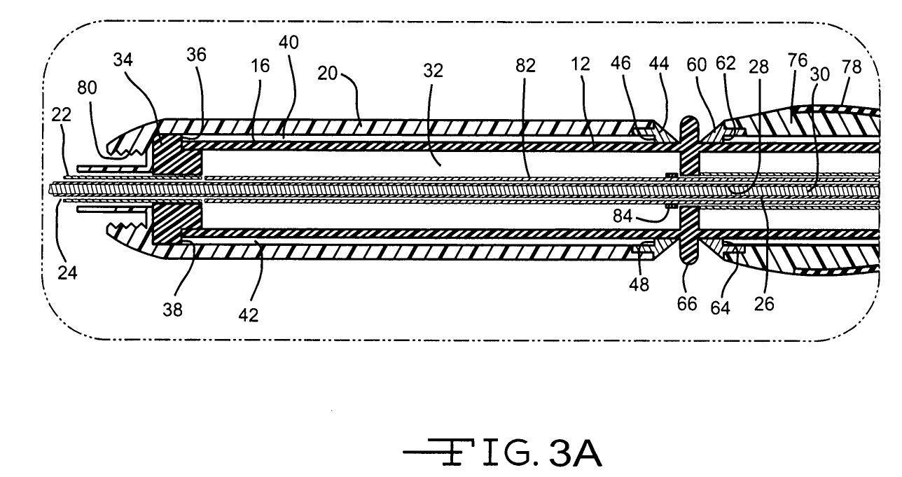

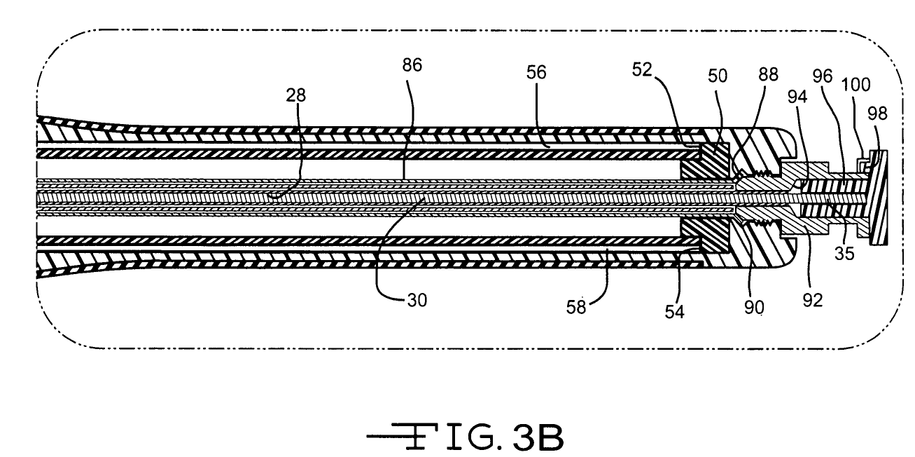

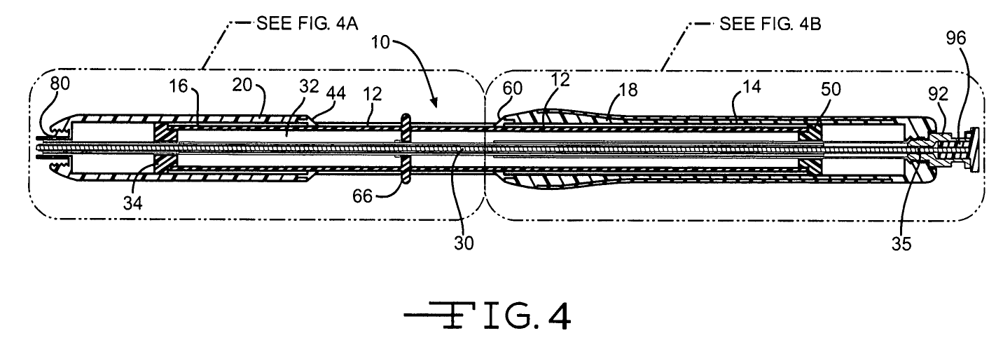

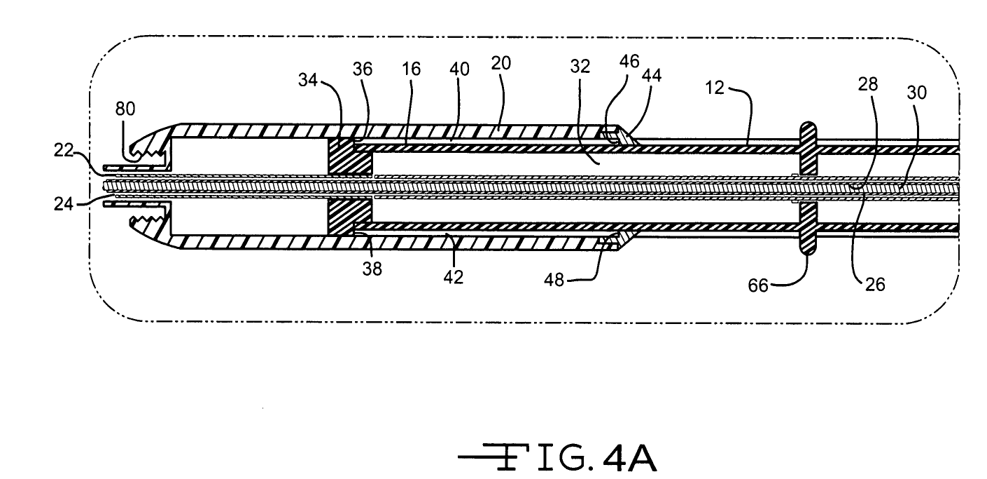

FIGS. 3 and 4 illustrate sectional views of the apparatus 10 in closed and open configurations, respectively. Together with the magnified views presented in FIGS. 3A, 3B, 4A and 4B, these figures illustrate the various mechanisms by which the apparatus 10 controls the relative positioning of the various components.

The inner handle member 12 defines a handle lumen 32 that contains various portions of various components of the apparatus 10. A distal cap 34 closes the handle lumen 32 at the distal end 16 of the inner handle member 12. The distal cap 34 can comprise an integral portion of the inner handle member 12 or can be a separately attached member. The distal cap 34 defines first 36 and second 38 distal cap stops. These stops 36, 38 are positioned at ends of first 40 and second 42 distal races defined by the inner handle member 12. The second outer handle member 20 includes a distal collar 44 that defines first 46 and second 48 projections. These projections 46, 48 move along the first 40 and second 42 distal races, respectively, as the second outer handle member 20 is slideably moved along the inner handle member 12. Eventually, the distal cap stops 36, 38 abut projections 46, 48 to prevent further distal movement of the second outer handle member 20. As with the distal cap 34, the distal collar 44 can comprise an integral component of the second outer handle member 20 or can be a separately attached member. Further, the inner handle member 12 can include a lesser or greater number of distal races, or the apparatus 10 generally can include any suitable alternative mechanism for limiting movement of the second outer handle member 20 along the inner handle member 12.

A proximal cap 50 closes the handle lumen 32 at the proximal end 14 of the inner handle member 12. The proximal cap 50 defines first 52 and second 54 proximal cap stops. These stops 52, 54 are positioned at ends of first 56 and second 58 proximal races defined by the inner handle member 12. The first outer handle member 18 includes a proximal collar 60 that defines first 62 and second 64 projections. These projections 62, 64 move along the first 56 and second 58 proximal races, respectively, as the first outer handle member 18 is slideably moved along the inner handle member 12. Eventually, the proximal cap stops 52, 54 abut projections 62, 64 to prevent further proximal movement of the first outer handle member 18. As with the distal cap 34 and collar 44, the proximal cap 50 and collar 60 can each comprises an integral component of the inner handle member 12 and the first outer handle member 18, respectively, or can be separate members attached to these components. Further, the inner handle member 12 can include a lesser or greater number of proximal races, or the apparatus 10 generally can include any suitable alternative mechanism for limiting movement of the first outer handle member 18 along the inner handle member 12.

The inner handle member 12 can also include a stop 66 disposed between the first 18 and second 20 outer handle members. The stop 66 separates the areas of the inner handle member 12 along which the first 18 and second 20 outer handle members can be moved. Further, the stop 66 provides a physical barrier to further movement of the outer handle members 18, 20 along the inner handle member 12. If present, the stop 66 can comprise an integral portion of the inner handle member 12, or can be a separately attached member. As best illustrated in FIG. 2, this stop 66 can comprise a circumferential projection defined by the inner handle member 12.

The apparatus 10 can further include various adaptations to facilitate operation of the apparatus 10. For example, as best illustrated in FIG. 2, a first series 68 of gradations 70 can be disposed on the inner handle member 12. If present, this series 68 can be disposed on a portion of the inner handle member 12 along which the first outer handle member 18 is moved. In this configuration, each gradation 70 of the series 68 can correspond to a predetermined position of the needle 26, which is attached to the first outer handle member 18, relative to a distal end of the sheath 22, which is attached to the inner handle member 12. Further, each gradation 70 of this series 68 can correspond to a predetermined length by which the needle 26 extends axially beyond a distal end of the sheath 22.

A further comparison of FIGS. 1 and 2 illustrates an example of the operation of this series 68 of gradations 70. In the open configuration illustrated in FIG. 2, the proximal most gradation viewable in the series 68 is “0”. Also in this configuration, the needle 26 does not extend beyond the distal end of the sheath 22. Thus, in this example, the gradation “0” can correspond to a zero length of the needle 26 that extends axially beyond a distal end of the sheath 22. In FIG. 1, the handle is in a completely closed configuration. To achieve this configuration from the open configuration illustrated in FIG. 2, a user would advance the first outer handle member 18 over the entire series 68 of gradations 70. As the user moves the first outer handle member 18 along the inner handle member 12, the first outer handle member 18 successively passes gradations 70 of the series 68. Each gradation 70 can correspond to a length by which the needle 26 extends beyond a distal end of the sheath 22. Once the first outer handle member 18 is fully advanced over the inner handle member 12, reaching stop 66, the entire series 68 of gradations 70 is covered. As illustrated in FIG. 1, this can correspond to a maximum length by which the needle 26 extends beyond the distal end of the sheath 22. Thus, by moving a distal end of the first outer handle member 18, such as collar 60, to a specific gradation 70 in the series 68, a user of the apparatus 10 can advance the needle 26 to a desired position relative to the sheath 22.

The apparatus 10 can also include a second series 72 of gradations 74. Similar to the first series 68, the second series 72 of gradations 74 can be disposed on the inner handle member 12. The second series 72 can be disposed on a portion of the inner handle member 12 along which the second outer handle member 20 is moved. In this configuration, each gradation 74 of the second series 72 can correspond to a predetermined length by which the sheath 22, which is attached to the inner handle member 12, extends axially beyond a distal end of the second outer handle member 20, which can be attached to another medical device.

A further comparison of FIGS. 1 and 2 illustrates an example of the operation of this series 72 of gradations 74. In the open configuration illustrated in FIG. 2, the proximal most gradation viewable in the series 72 is “0”. The gradation “0” can refer to a particular length by which the sheath 22 extends beyond a distal end of the second outer handle member 20. If the second outer handle member 20, and thus the entire apparatus 10, is used with another medical device having a working lumen, such as an endoscope, the gradation “0” can correspond to a zero length of the sheath 22 that extends axially beyond a distal end of the other medical device. For example, the “0” gradation may indicate that no portion of the sheath 22 extends out of the working lumen of the attached medical device. The second outer handle member 20 passes successive gradations 74 in the series 72 as it is moved along the inner handle member 12. Each gradation 74 in the series 72 can correspond to a predetermined length by which the sheath 22 extends beyond a distal end of the second outer handle member 20. Further, if the second outer handle member 20 is used with another medical device, each gradation 74 can correspond to a predetermined length by which the sheath extends axially beyond a distal end of the medical device. Once the second outer handle member 20 is fully advanced over the inner handle member 12, reaching stop 66 and the closed configuration illustrated in FIG. 1, the entire series 72 of gradations 74 is covered, which can indicate a maximum length by which the sheath 22 extends axially beyond the distal end of the second outer handle member 20 or a distal end of an attached medical device.

The first outer handle member 18 can include structural adaptations that facilitate operation of the apparatus 10. For example, the first outer handle member 18 can define an enlargement 76 that provides a resting position for a finger or thumb of the user. The enlargement 76 represent a circumferential portion of the first outer handle member 18 that has a larger outer diameter than another portion of the first outer handle member 18. Further, a grip insert 78 can be attached to or defined by the first outer handle member 18. The grip insert 78 provides a surface that facilitates handling of the apparatus 10. The grip insert 78 can be formed of the same material as the first outer handle member 18, such as a plastic, or can comprise a different material, such as a rubber or other polymeric material.

The apparatus 10 can be used with other medical devices. In some embodiments, it may be desirable to allow attachment of the apparatus 10 to another medical device. The second outer handle member 20 can define structural adaptations that facilitate attachment of the apparatus 10 to another medical device. For example, the second outer handle member 20 can define a connector 80. The connector 80 is structurally capable of interacting with another connector on the other medical device to which the apparatus 10 is to be attached. This interaction between the connector 80 and the other connector on the medical device can be a mating connection, and can be a locking connection. Any suitable connector can be used as the connector 80, and a Luer-type connector is an example of a particularly well suited connector. Other suitable types of connectors include clamp connectors and engagement member connections, such as thumb screws and the like.

The apparatus 10 can include additional components that facilitate the relative movement of the interior components of the device. Examples of such additional components are illustrated in FIGS. 3A, 3B, 4A, and 4B. An inner guide tube 82 can be disposed in the handle lumen 32 and around the needle 26. The inner guide tube 82 is a tubular member that surrounds the needle 26. The inner guide tube 82 can define a collar 84 disposed near the stop 66 of the inner handle member 12. The inner guide tube 82 can be attached to the inner handle member 12. Also, as best illustrated in FIG. 3A, the position of the inner handle member 12 can be fixed by its surrounding the needle 26, the positioning of the collar 84 adjacent the stop 66, or both. As best illustrated in FIG. 3B, an outer guide tube 86 can also be disposed in the handle lumen 82 and around a portion of the inner guide tube 82. In this embodiment, the outer guide tube 82 is attached to the first outer handle member 18 and, therefore, slideably moves along the inner guide tube 82 as the first outer handle member 18 is slideably moved along the inner handle member 12. The attachment of the outer guide tube 86 to the first outer handle member 18 can be accomplished in any suitable manner. In this embodiment, the outer guide tube 86 defines a flare 88 at a proximal end. The flare 88 is disposed in a recess 90 of the first outer handle member 18. An access port 92 is positioned at a proximal end of the first outer handle member 18 and adjacent the flare 88, effectively locking the outer guide tube 86 in position relative to the first outer handle member 18. This attachment can also include an adhesive or other suitable bonding mechanism.

The inclusion of inner 82 and outer 86 guide tubes may prevent buckling of components within the handle lumen 32 during repetitive movement of the apparatus 10 between open and closed configurations.

The access port 92 provides access to the needle lumen 28 from an environment external to the apparatus 10. The access port 92 can be integrally formed by the first outer handle member 18 or can comprise a separately attached member.

The stylet 30 is slideably disposed in the access port 92. The stylet 30 includes a cap 96 at the proximal end of the first outer handle member 18 which is attached to the stylet's proximal end 35 which facilitates insertion and removal from the access port 92. The cap 96 can interact with a throat 94 defined by the access port 92 to guide and/or limit the movement of the stylet 30 into the access port 92. Further, the cap 96 can define structural adaptations that fix the position of the cap 96 relative to the access port 92. For example, the cap 96 can define a notch 98 that is received by a slot 100 defined by the access port 92. This interaction between the notch 98 and the slot 100 may prevent rotational and axial movement of the cap 96 and the attached stylet 30.

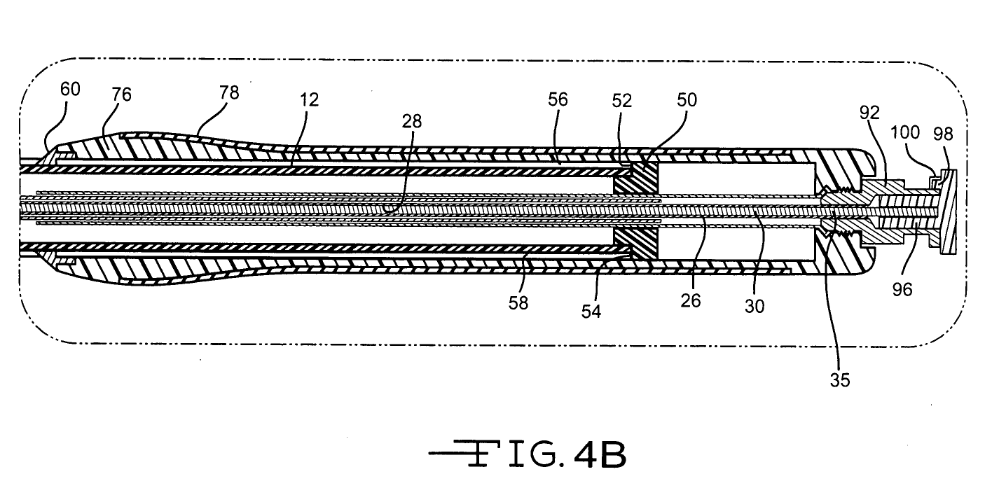

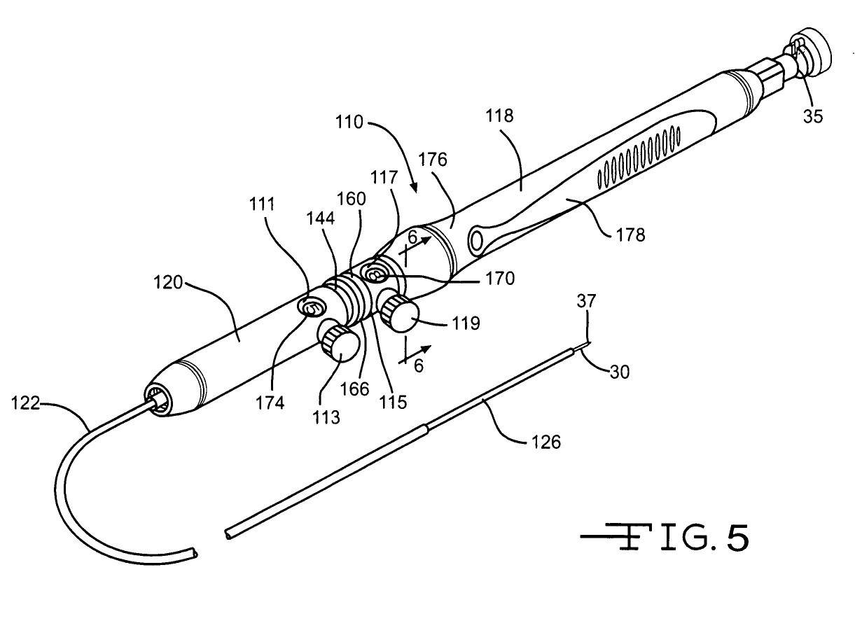

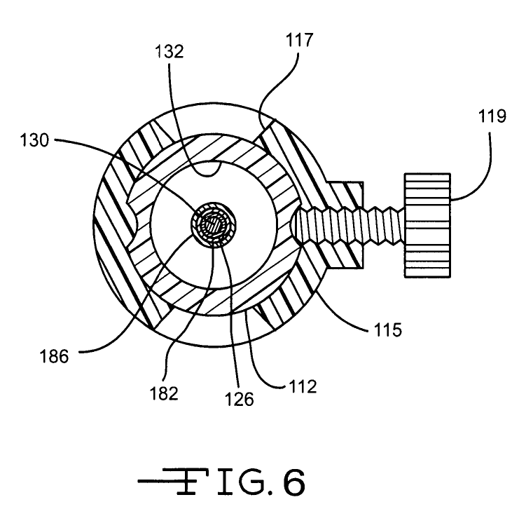

FIGS. 5 and 6 illustrate a handle 110 according to another embodiment of the invention. The handle 110 of this embodiment is identical to the handle of the embodiment described above, except as detailed below. Thus, like reference numbers in FIGS. 5 and 6 refer to similar features and/or components of the embodiment described above and illustrated in FIGS. 1-3, 3A, 3B, 4, 4A, and 4B. FIG. 5 illustrates the handle 110 according to this embodiment in a closed configuration.

In this embodiment, the second outer handle member 120 defines an aperture 111. The aperture 111 is positioned on the second outer handle member 120 such that it is disposed over a portion of a series of gradations 174 disposed on the inner handle member 112. The size and configuration of the aperture 111 can vary, but should be such that the aperture 111 can reveal one or more of the gradations 174 in a meaningful manner.

In this embodiment, the second outer handle member 120 also includes a means for fixing an axial position of the inner handle member 112 relative to the second outer handle member 120. Any suitable means for fixing an axial position between two slideably engaged components can be used. For example, a selectively engageable member that extends through a thickness of the second outer handle member 120 can be used as the means for fixing. The selectively engageable member can be withdrawn from the thickness or advanced through the thickness to engage the inner handle member 112. When disposed through the thickness and in contact with the inner handle member 112, the axial position of the inner handle member 112 relative to the second outer handle member 120 becomes fixed. That is, further axial movement of the second outer handle member 120 along the inner handle member 112 is hindered because of the contact between the inner handle member 112 and the selectively engageable member.

FIG. 5 illustrates a thumb screw 113 that is a suitable selectively engageable member for use as the means for fixing. The thumb screw 113 is readily advanced through a thickness of the second outer handle member 120 and into contact with the inner handle member 112 to fix a relative axial position between the inner 112 and second outer handle 120 members. The second outer handle member 120 provides a thread that interacts with a complimentary thread on the thumb screw 113 to allow its extension into and out of the thickness of the second outer handle member 120.

In this embodiment, a slideable member 115 is also disposed on the inner handle member 112. The slideable member 115 can be a locking member that is slideably disposed on the inner handle member 112. The slideable member 115 can include structural adaptations that allow it to be locked at any of a plurality of positions on the inner handle member 112. For example, the slideable member 115 can include a means for fixing as described above, such as a thumb screw 119 as described above for the second outer handle member 120. The slideable member 115 provides a movable stop that limits movement of the first outer handle member 118 along the inner handle member 112. This stop, therefore, limits the movement of the needle 126, which is attached to the first outer handle member 118, relative to the sheath 122. By locking the slideable member 115 at a desired position along the inner handle member 112, which may be indicated by a gradation 170, a user of the handle 110 can set a maximum length by which the needle 126 can extend beyond the sheath 122. Thus, a user can move the first outer handle member 118 along a span of the inner handle member 112 between the fully retracted position and the position at which the slideable member 115 is locked. This span can be a limited portion of the inner handle member 112 along which the first outer handle member can be move, and the precise length of the span depends on the position at which the slideable member 115 is locked. In turn, the chosen position for the slideable member 115 will depend on the desired maximum extension length of the needle 126 relative to the sheath 122.

To facilitate the positioning of the slideable member 115 at desired locations on the inner handle member 112, the slideable member 115 can define an aperture 117 that reveals an underlying portion of the inner handle member 112, which may include one or more gradations 170. The aperture 117 can take any suitable size, shape, and configuration, but should be adapted to reveal an underlying portion of the inner handle member in a meaningful manner, such as at least one complete gradation 170. As illustrated in FIGS. 5 and 6, the slideable member 115 can comprise a collar that is circumferentially disposed around the inner handle member 112 and between the first 118 and second 120 outer handle members.

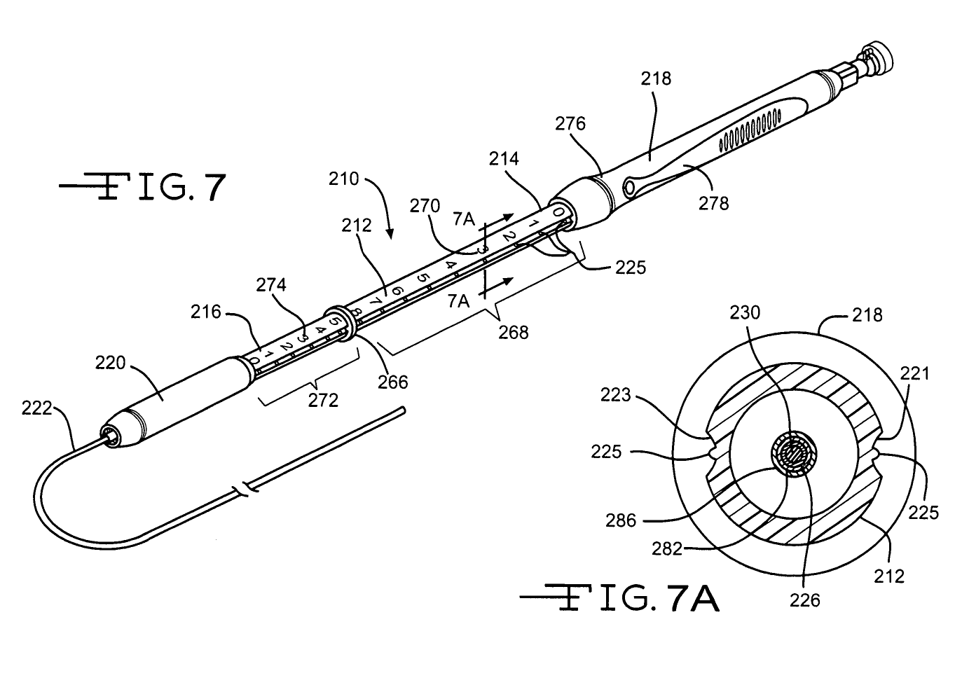

FIG. 7 and 7A illustrate a handle 210 according to another embodiment of the invention. The handle 210 according to this embodiment is identical to the handle of the first embodiment described above, except as detailed below. Thus, like reference numbers in FIGS. 7 and 7A refer to similar features and/or components of the embodiment described above and illustrated in FIGS. 1-3, 3A, 3B, 4, 4A, and 4B. FIG. 7 illustrates the handle according to this embodiment in an open configuration.

In this embodiment, the inner handle member 212 defines first 221 and second 223 races. One or both of the races 221, 223 include a plurality of stops 225 that define discrete positions on the handle member at which another component, such as the outer handle members 218, 220 or another slideably attached member, can be disposed. In this embodiment, the stops 225 comprise projections defined by the inner handle member 212 and disposed in the races 221, 223. The stops 225 can also comprise separately attached members disposed in the races 221, 223.

The stops 225 temporarily stop slideable movement of a component over the inner handle member 212, but do not halt such movement completely. Rather, the stops 225 simply provide resistance that can be overcome by additional force to produce continued slideable movement of the component along the inner handle member 212. The slideable component can interact with the stops 225 to produce a sound when the slideable component is moved along the inner handle member 212. This production of a sound can provide additional feedback to an operator of the handle 210 that indicates relative position of various components of the handle 210. The slideable components that interact with the stops 225 in this manner could be one or both of the outer handle members 218, 220, or any other slideable component disposed on the inner handle member 212, such as the slideable member described above in relation to the embodiment illustrated in FIGS. 5 and 6.

The stops 225 can be positioned in any suitable arrangement and configuration on the inner handle member 212. As best illustrated in FIG. 7, the stops 225 can be disposed adjacent each gradation 270 in a series of gradations 268 disposed on the inner handle member 212. Also, a first set of stops 225 can be disposed on one portion of the inner handle member 212 while a second set of stops 225 can be disposed on a second portion of the inner handle member 212. For example, as illustrated in FIG. 7, a first set of stops 225 can be disposed adjacent the gradations 270 of a first series 268 of gradations, and a second set of stops 225 can be disposed adjacent gradations 274 of a second series 272 of gradations.

In this embodiment, the inner handle member can define any suitable number of races, and one or more of the races can include stops 225.

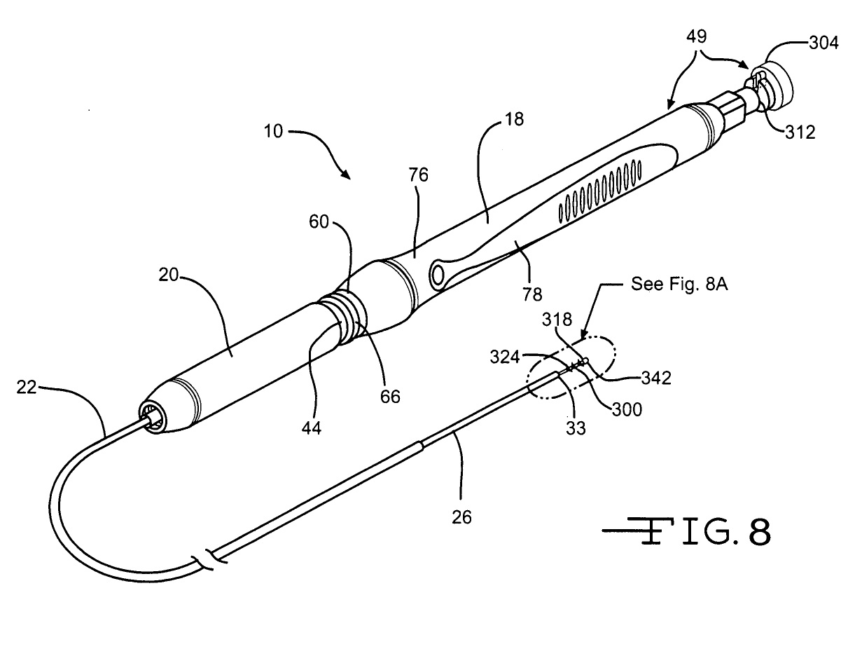

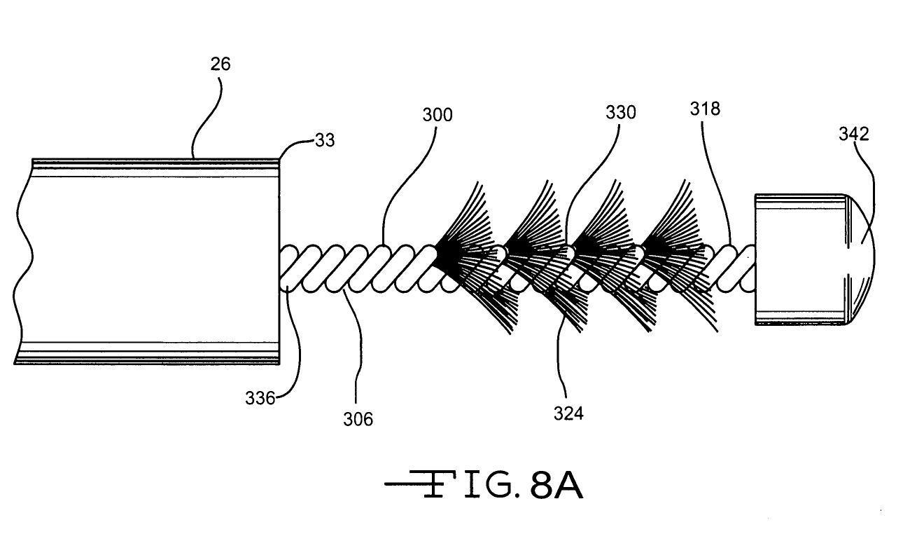

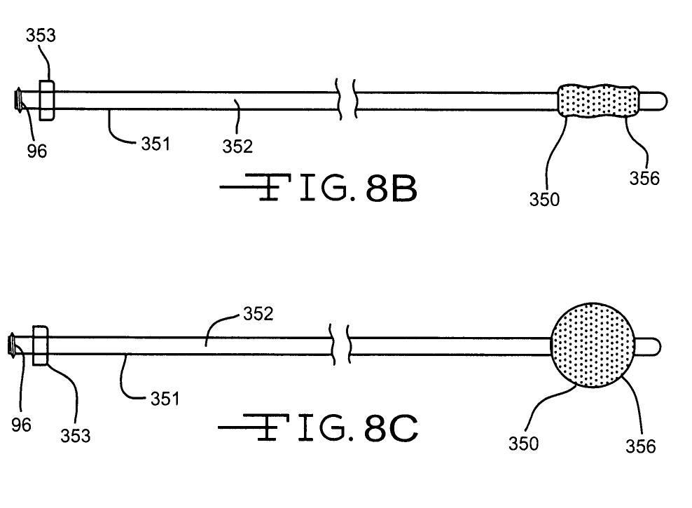

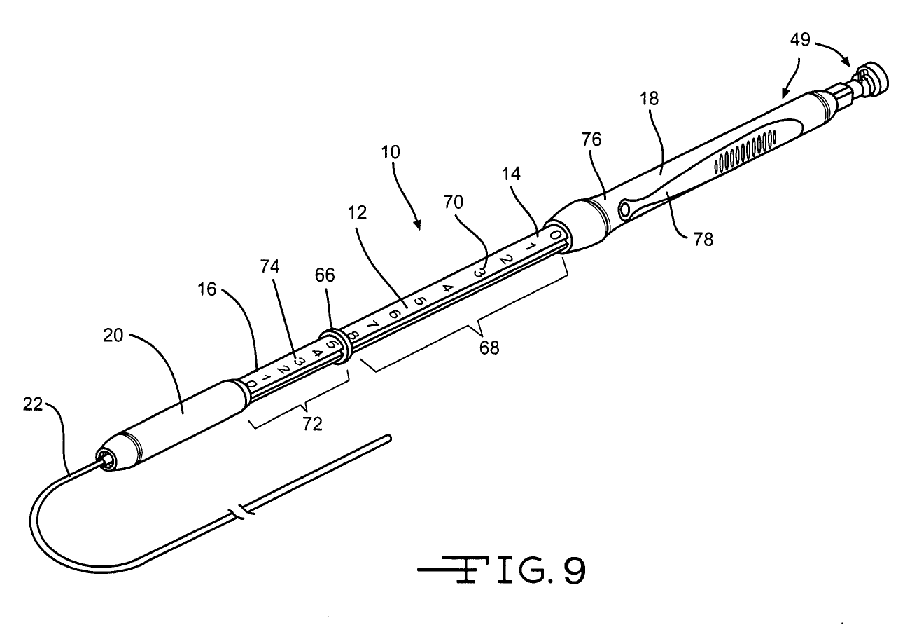

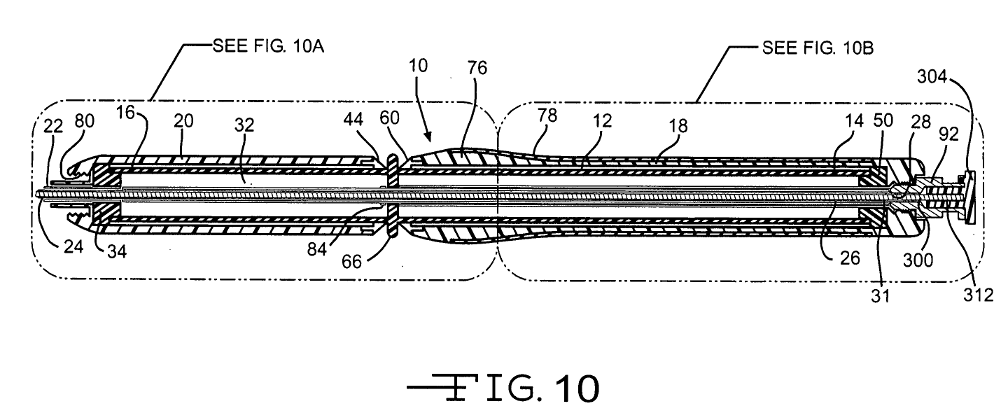

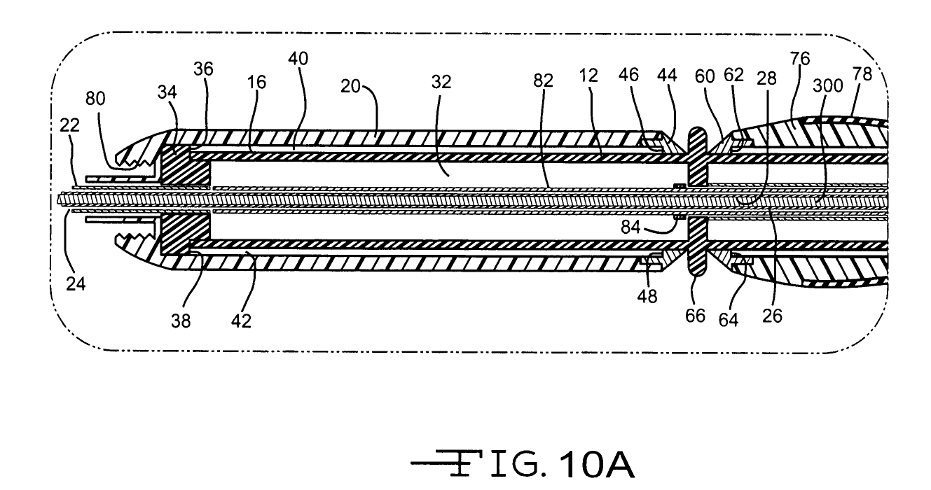

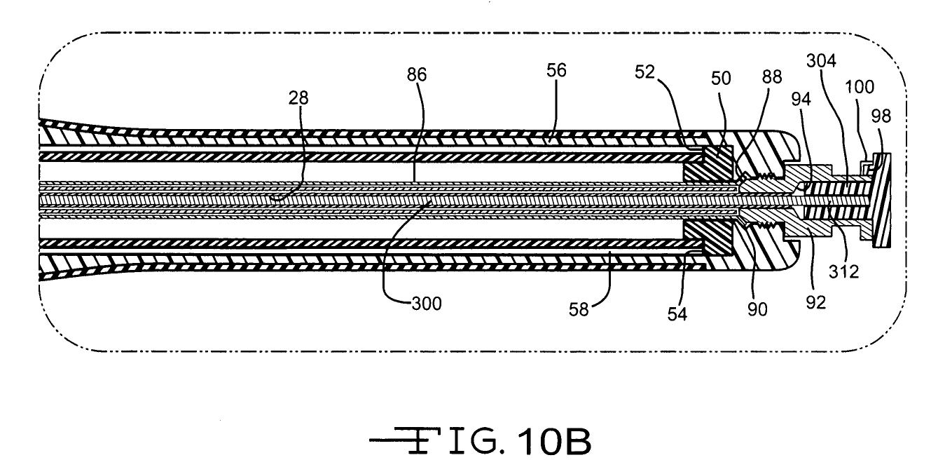

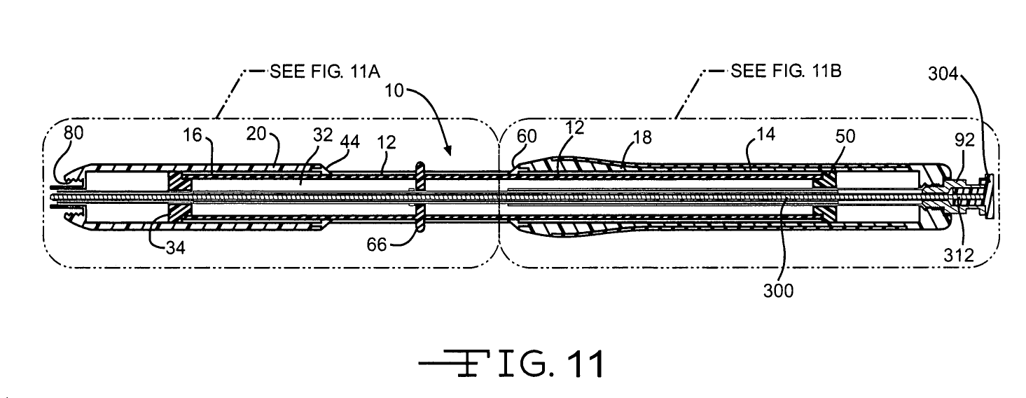

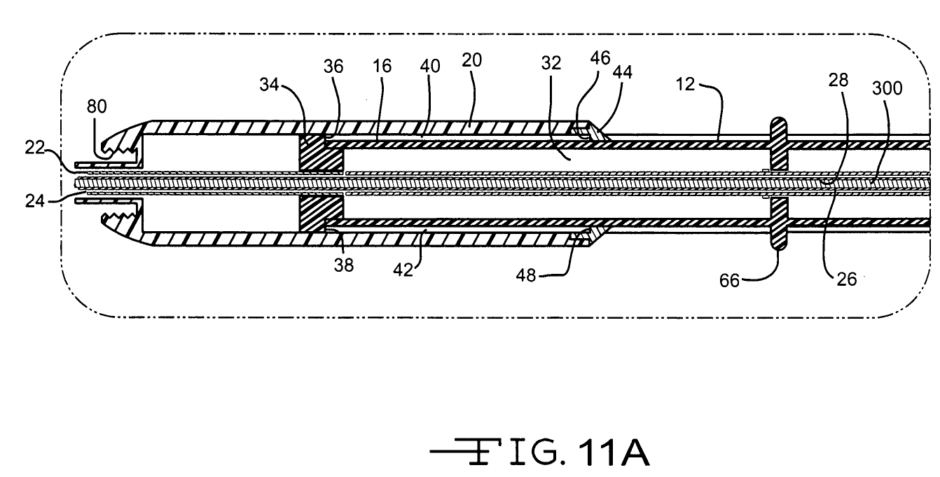

FIGS. 8, 8A, 9-10, 10A, 10B, 11, 11A, and 11B illustrate the apparatus identically as shown and discussed previously in FIGS. 1, 1A, 2-3, 3A, 4, 4A, and 4B except the stylet 30 has been withdrawn from the needle lumen 28 and a brush 300 has been inserted in its place within the needle lumen 28. The brush 300 is a cytology collection device which may be utilized to collect the cytology sample previously cut by the stylet 30. In other embodiments, differing cytology collection devices may be used such as an inflatable balloon, a wire mesh device, or other types of collection devices. When inserted into the needle lumen 28, the brush 300 may be at various times during the procedure partially disposed in the needle lumen 28, or entirely disposed within the needle lumen 28. The brush 300 comprises an elongate member 306, having a proximal end 312, a distal end 318, and a plurality of bristles 324 connected to the elongate member 306 at a location 330 near the distal end 318 of the elongate member 306. The elongate member 306 comprises a wire 336. The wire 336 may be a variety of materials including steel, other types of metals, and nitinol. The wire 336 is twisted around the plurality of bristles 324 to connect the plurality of bristles 324 to the wire 336. The bristles 324 may be a variety of materials including nylon, brass, steel, carbon, polymer, or other types of metals. The brush 300 extends axially beyond the second outer handle member 20, and into the sheath lumen 24. The distal end 318 of the elongate member 306 terminates in a steel cap 342. In other embodiments, the elongate member 306 may terminate in a loop, ball tip, cone tip, bevel tip, or other types of tips, and the tip may be a variety of materials including different types of metal and solder. As discussed, the handle 49 is adapted to provide axial movement of the brush 300 and needle 26. In addition to the preferred embodiment as described, the handle 49 may comprise a variety of types including a finger-loop handle, a pin-vice handle, and a half-finger loop handle.

The brush 300 is slideably disposed in the access port 92. The brush 300, like the stylet 30, includes a cap 304 at the proximal end of the first outer handle member 18 which is attached to the elongate member 306 which facilitates its insertion into and removal from the access port 92. The cap 304 can interact with a throat 94 defined by the access port 92 to guide and/or limit the movement of the brush 300 into the access port 92. Further, the cap 304 can define structural adaptations that fix the position of the cap 304 relative to the access port 92. For example, the cap 304 can define a notch 98 that is received by a slot 100 defined by the access port 92. This interaction between the notch 98 and the slot 100 may prevent rotational movement of the cap 304 and the attached brush 300.

Initially, the brush's distal end 318 is disposed within the needle's inner lumen 28. The cap 304 is then extended axially towards the handle 49 in order to extend the brush's distal end 318 outside of the needle's inner lumen 28. Once the brush's distal end 318 is extended outside the needle's inner lumen 28, the cap 304 is moved back and forth axially in order to collect a cytology sample utilizing the bristles 324 near the brush's distal end 318. Once the cytology sample has been obtained, the cap 304 is retracted axially away from the handle 49 in order to retract the brush's distal end 318 within the needle's inner lumen 28 to prevent the sample from being contaminated by unwanted liquids or substances during removal of the sample from the body. The cap 304 may abut against the needle's distal end 33 to prevent the sample from being contaminated. A mark or marker may be used near the proximal end 312 of the brush 300 to indicate to the operator when the distal end 318 of the brush 300, or other cytology collection device, is extended beyond the needle's distal end 33.

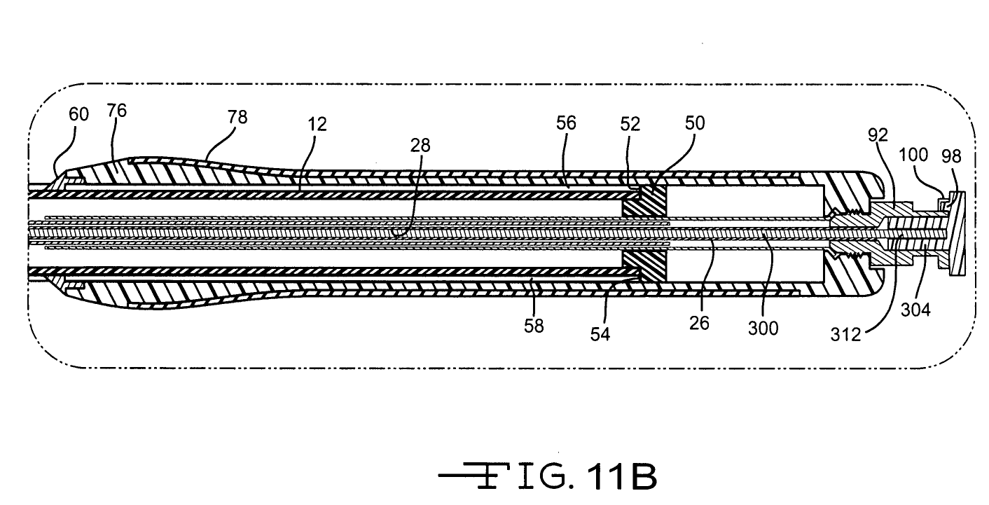

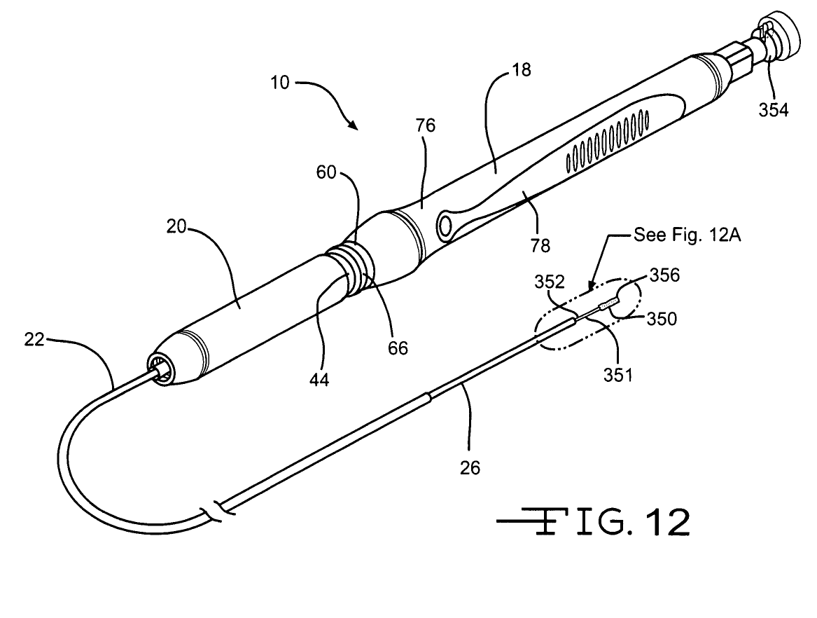

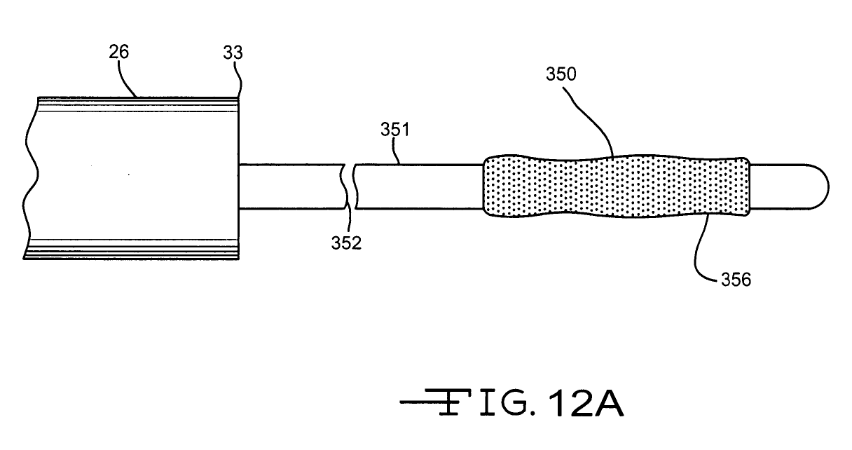

FIGS. 12, 12A, 12B, and 12C illustrate another embodiment in which the brush 300 of the above embodiment is replaced with an inflatable and deflatable balloon 350 acting as the cytology collection device utilized to collect the cytology sample previously cut by the stylet 30. The balloon 350 is connected to a tube 351 having an inner lumen 352 through which air or liquid can be injected to fill the balloon 350. The tube 351 and balloon 350 assembly run through the inner handle 12, into the sheath lumen 24, and into the inner lumen 28 of the needle 26. A cap 354 at the end of the tube 351 may screw into place to lock the tube in position. The balloon 350 is adapted to fit within the inner lumen 28 of the needle 26 when deflated and is adapted to inflate when outside of the inner lumen of the needle 28 in order to collect cytology samples within a mammalian body. FIG. 12B shows the balloon 350 and tube 351 assembly with the balloon 350 in a deflated position. FIG. 12C shows the balloon 350 and tube assembly 351 with the balloon 350 in an inflated position. An outer surface 356 of the balloon 350 is rough in order to abrade against cell tissue to collect a cytology sample when inflated. The balloon 350 may be made of a variety of materials including polymer, silicone, and polyethylene terephthalate. The cap 354 is slideably disposed at the proximal end of the first outer handle member 18 and is attached to the balloon 350 and tube 351 assembly in order to control axial movement of the balloon 350. Using the cap 354 to control axial movement, the balloon 350 is initially inside the inner lumen 28 of the needle 26 when deflated, slid outside the inner lumen 28 of the needle 26 to be inflated in order to obtain a cytology sample, and then retracted within the inner lumen 28 of the needle 26 after obtaining the cytology sample.

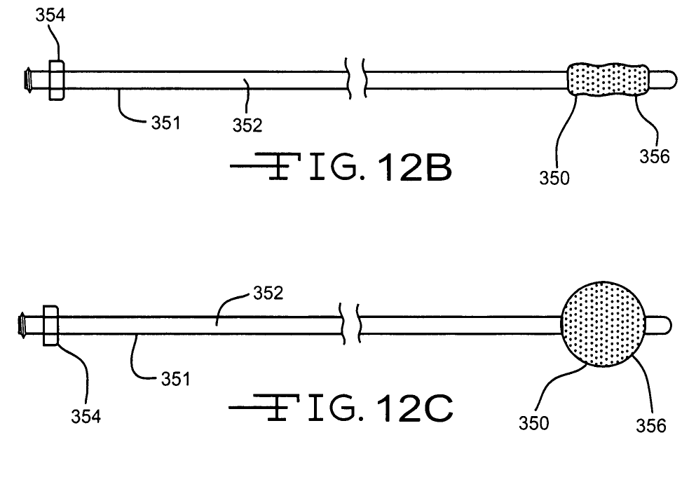

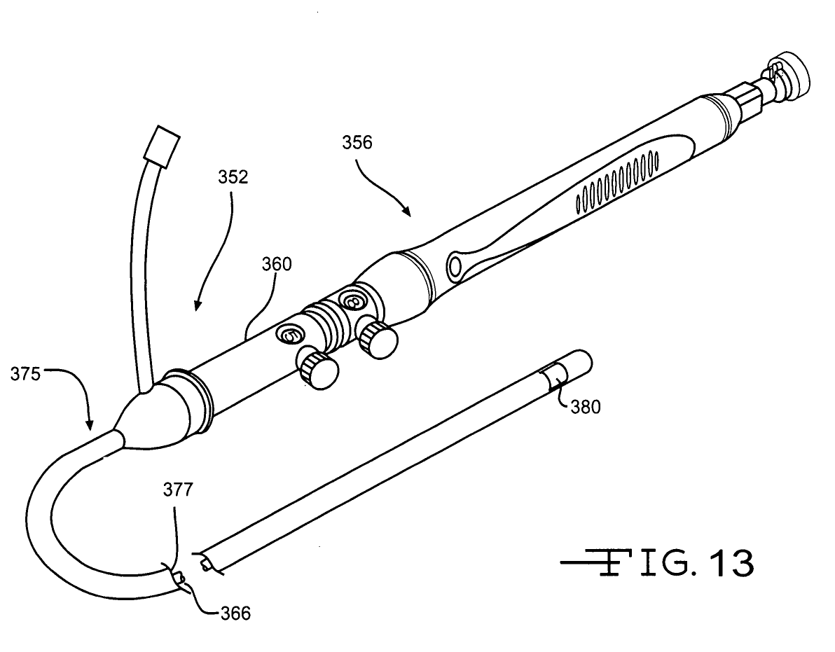

FIG. 13 illustrates a medical device assembly 352 according to another embodiment of the invention. The medical device assembly 352 comprises a handle 356 according to any embodiment of the invention. The handle 356 is attached to a medical device 375. The medical device 375 defines a working lumen 377. The sheath 366 of the handle 356 is attached to the inner handle member of the handle 356 and axially extends beyond the distal end of the inner handle member and into the working lumen 377 of the medical device 375. The attachment of the handle 356 to the medical device 375 can be accomplished in any suitable manner, including a connector disposed on the second outer handle member 360 as described above.

A suitable medical device 375 for use in the medical device assembly 352 according to the invention comprises an endoscope. In any of the embodiments described above, an endoscope may be included containing a transducer 380 which emits a series of waves or a pulse. The waves or pulse propagate through the body tissue in the mammalian body at a speed that is determined by the physical properties of the tissue. The waves or pulse are then reflected back to the transducer 380 when the sound waves encounter a tissue or other mass which is more difficult to pass through. The transducer 380 detects the reflected waves and translates them into electrical signals for processing an image in order to determine the locations of the tissue or other masses upon which the waves reflected. In such manner, the transducer 380 may determine a position of a cytology collection device, such as a balloon or a brush, within the body. In one embodiment, to aid the reflection of the ultrasound waves, the cytology collection device may comprise a dimpled wire having a proximal end, a distal end, and a plurality of bristles connected to the dimpled wire at a location near the distal end of the wire. The dimpled wire helps reflect the ultrasound waves. In another embodiment, the bristles may be steel or another material which helps aid the reflection of the ultrasound waves. Use of this apparatus and method may aid the operator or physician in directing the cytology collection device within the body during the procedure.

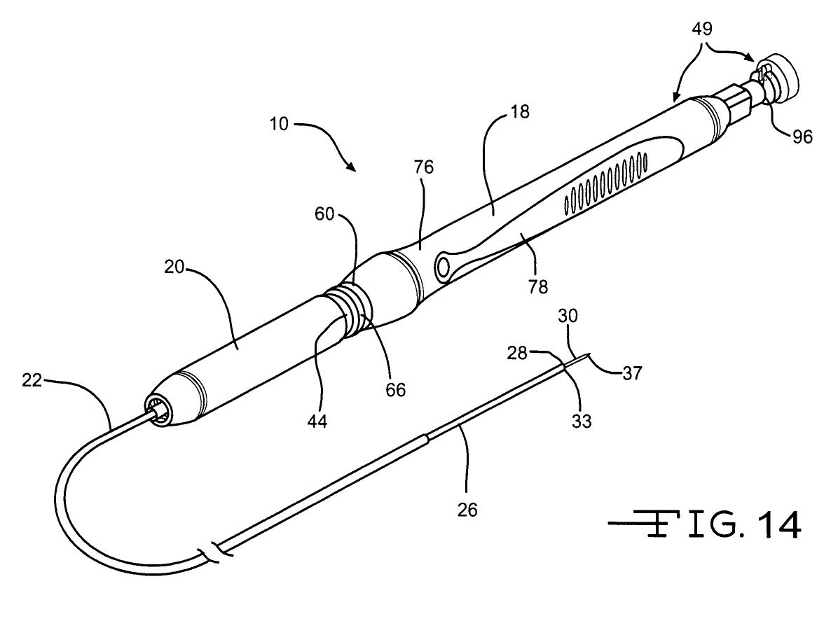

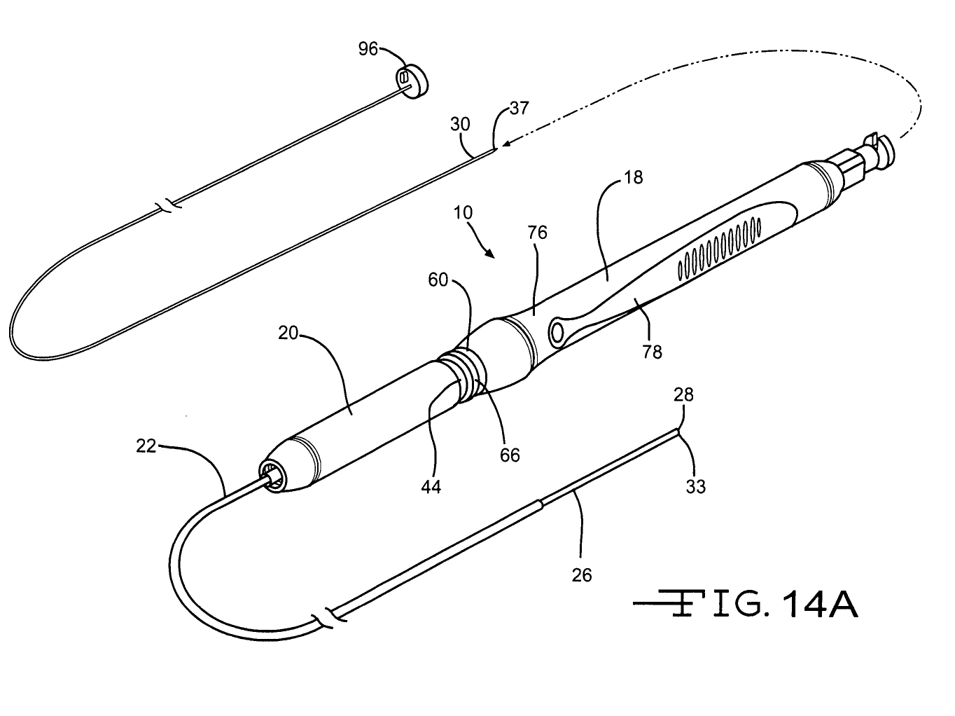

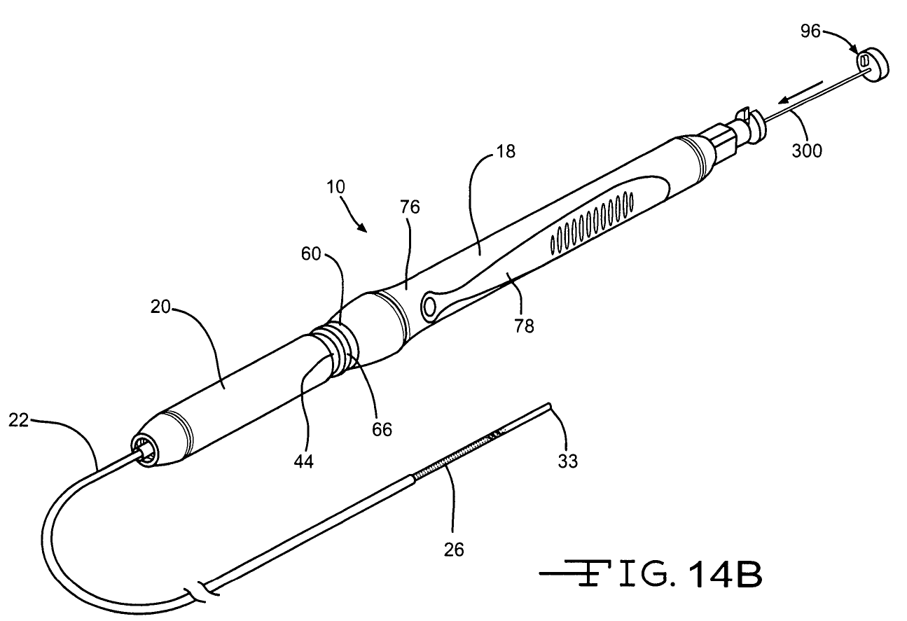

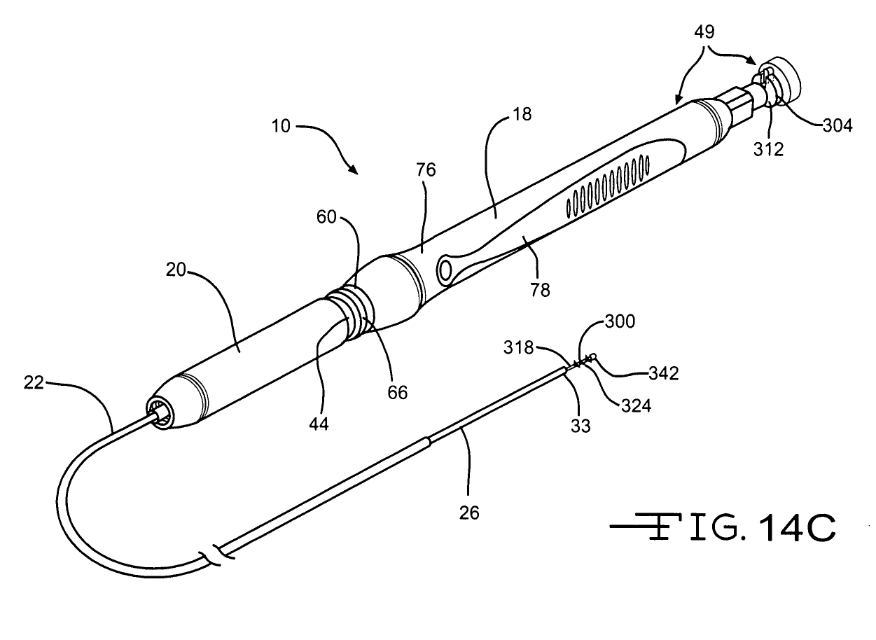

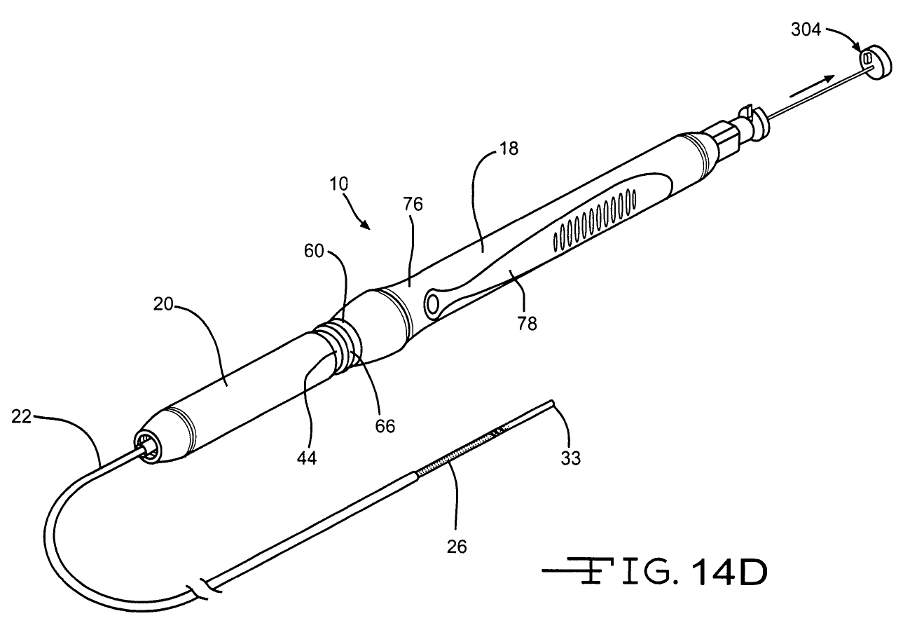

Using any of the above disclosed embodiments, a multitude of methods may be employed to take a cytology sample from a mammalian body. FIGS. 14-14D disclose the steps of one such method using the apparatus of FIGS. 1-4B and FIGS. 8-11B. As shown in FIG. 14, the stylet 30 is inserted into the inner lumen 28 of the needle 26 with the sharp distal end 37 of the stylet 30 extending beyond the distal end 33 of the needle 26. The stylet 30 plugs the inner lumen 28 of the needle 26 in order to avoid contamination during cutting of a sample. Then, an area within the mammalian body, such as a cyst, is cut using the sharp distal end 37 of the stylet 30 by moving the cap 96, attached to the proximal end 35 of the stylet 30, back and forth axially. In other embodiments and/or methods, the distal end 37 of the stylet 30 may be dull, it may not be adapted to cut, and its sole purpose may be to plug the inner lumen 28 of the needle 26 during the cutting procedure. In those embodiments, the needle 26 or other apparatus and/or methods known in the art may be utilized to cut an area within the mammalian body. Next, as shown in FIG. 14A, the stylet 30 is withdrawn from the inner lumen 28 of the needle 26. At this point, suction may be applied through the needle 26. Then, as shown in FIG. 14B, the cytology collection device 300 is inserted into the inner lumen 28 of the needle 26. In other embodiments, the apparatus may include an endoscope containing a transducer emitting ultrasound waves which may be used to determine a position of the cytology collection device within the body. Next, as shown in FIG. 14C, the distal end 318 of the cytology collection device 300 is extended until the cytology collection device 300 extends beyond the distal end 33 of the needle 26. A cytology sample is then collected using the cytology collection device 300 by moving the cap 304, attached to the proximal end 312 of the cytology collection device 300, back and forth axially. Finally, as shown in FIG. 14D, the distal end 318 of the cytology collection device 300 is retracted into the inner lumen 28 of the needle 26 in order to avoid contamination of the collected sample. The apparatus is then removed from the body.

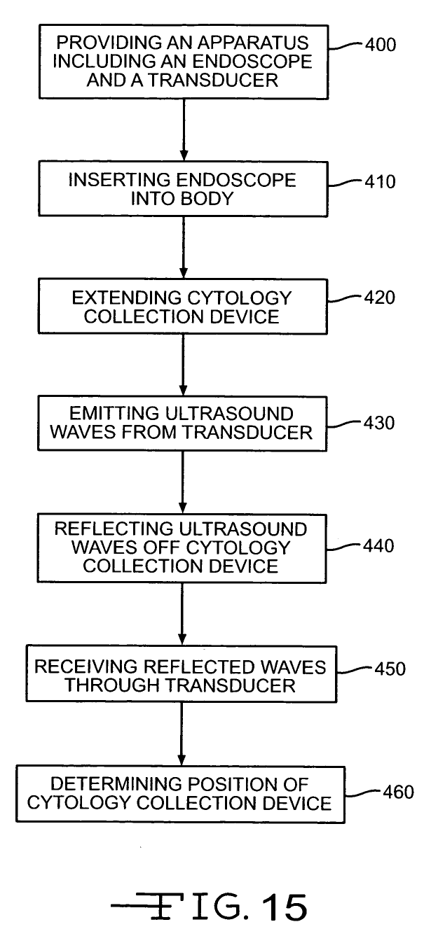

FIG. 15 depicts, in flowchart format, another method for taking a cytology sample from a mammalian body. First, an apparatus is provided 400. The apparatus includes an endoscope which defines a working lumen and contains a transducer. Additionally, the apparatus includes a first member containing a proximal end, a distal end, and an inner lumen, which extends into the working lumen of the endoscope. The first member is preferably a needle. Moreover, the apparatus also includes a cytology collection device having a proximal end, and a distal end for cytology collection, which extends into the inner lumen of the first member. The working lumen of the endoscope is inserted into a mammalian body 410. Next, the distal end of the cytology collection device is extended beyond the distal end of the first member 420. Ultrasound waves are emitted from the transducer of the endoscope 430. The ultrasound waves are reflected off the cytology collection device 440, due to its material and composition, and received by the endoscope's transducer 450. The transducer translates the waves into electronic signals in order to form images. Finally, a position of the cytology collection device within the mammalian body is determined from the translated images formed from the ultrasound waves received by the transducer 460.

The forgoing detailed description provides exemplary embodiments of the invention and includes the best mode for practicing the invention. These embodiments are intended only to serve as examples of the invention, and not to limit the scope of the invention in any manner.

We claim:

1. A medical apparatus comprising:
a needle having a proximal end, a distal end, and an inner lumen extending from said proximal end to said distal end;
a stylet having a proximal end and a distal end, wherein said stylet is adapted to be inserted into and withdrawn from said inner lumen of said needle with at least a portion of said stylet adapted to plug said inner lumen of said needle when a cytology sample is cut; and
a cytology collection device having a proximal end and a distal end, wherein said cytology collection device is adapted to be inserted into said inner lumen of said needle when said stylet is withdrawn from the inner lumen of said needle, with said distal end of said cytology collection device adapted to extend beyond the distal end of said needle in order to collect said cytology sample.

2. An apparatus according to claim 1, wherein said cytology collection device comprises an elongate member having a proximal end, a distal end, and a plurality of bristles.

3. An apparatus according to claim 1, wherein said distal end of said cytology collection device terminates in one of a cap, ball tip, cone tip, bevel tip, and loop.

4. An apparatus according to claim 3, wherein one of said cap, ball tip, cone tip, bevel tip, and loop is made of one or more of steel, metal, and solder.

5. An apparatus according to claim 2, wherein said elongate member comprises a wire and said wire is twisted around said plurality of bristles.

6. An apparatus according to claim 5, wherein said wire is made of one or more of steel, metal, and nitinol.

7. An apparatus according to claim 2, wherein the distal end of the elongate member is adapted to be retracted within the inner lumen of said needle after collecting said cytology sample.

8. An apparatus according to claim 1, wherein said needle is made of one or more of steel, and metal.

9. An apparatus according to claim 2, wherein said plurality of bristles is made of one or more of nylon, brass, stainless steel, metal, carbon, and polymer.

10. An apparatus according to claim 1, wherein said needle is adapted to provide suction.

11. An apparatus according to claim 1, further comprising a handle which is adapted to provide axial movement of said cytology collection device and said needle.

12. An apparatus according to claim 11, wherein said handle comprises:
an inner handle member having a proximal end and a distal end;
a first outer handle member slideably disposed on the inner handle member; and
an elongate sheath attached to the inner handle member and axially extending beyond the distal end of the inner handle member, the sheath defining a sheath lumen, wherein said needle is attached to the first outer handle member and disposed in the sheath lumen.

13. An apparatus according to claim 11, wherein said handle comprises:
an inner handle member having proximal and distal ends and defining a handle lumen;
a first outer handle member slideably disposed on the proximal end of the inner handle member;
a second outer handle member slideably disposed on the distal end of the inner handle member;
an elongate sheath attached to the inner handle member and axially extending beyond the distal end of the inner handle member, the sheath defining a sheath lumen, wherein said needle is attached to the first outer handle member, the needle extending through the handle lumen and into the sheath lumen; and
a cap slideably disposed at the proximal end of the first outer handle member, said cap connected to the cytology collection device.

14. An apparatus according to claim 13, further comprising an endoscope defining a working lumen, wherein said elongate sheath axially extends into the working lumen of the endoscope.

15. An apparatus according to claim 11, wherein said handle comprises one of a finger-loop handle, a pin-vice handle, and a half-finger loop handle.

16. An apparatus according to claim 1, further comprising an endoscope containing a transducer which is adapted to emit ultrasound waves to determine a position of said cytology collection device within a mammalian body using said ultrasound waves.

17. An apparatus according to claim 16, wherein said cytology collection device comprises a dimpled wire having a proximal end, a distal end, and a plurality of bristles, wherein said transducer is adapted to emit ultrasound waves capable of reflecting off said dimpled wire.

18. An apparatus according to claim 16, wherein said cytology collection device comprises an elongate member having a proximal end, a distal end, and a plurality of bristles, wherein said transducer is adapted to emit ultrasound waves capable of reflecting off said plurality of bristles.

19. An apparatus according to claim 18, wherein said plurality of bristles is made of one or more of nylon, brass, stainless steel, metal, carbon, and polymer.

20. An apparatus according to claim 1, wherein said stylet is a solid rod.

21. An apparatus according to claim 1, wherein said stylet is made of one or more of steel, metal, and nitinol.

22. An apparatus according to claim 1, wherein said cytology collection device comprises a balloon adapted to fit within said inner lumen of said needle when deflated and adapted to inflate when outside of said inner lumen of said needle in order to collect said cytology sample.

23. An apparatus according to claim 22, wherein an outer surface of said balloon is rough.

24. An apparatus according to claim 22, wherein said balloon is made of one or more of polymer, silicone, and polyethylene terephthalate.

25. An apparatus according to claim 1, further comprising a marker near said proximal end of said cytology collection device which is adapted to indicate when said distal end of said cytology collection device has been extended beyond the distal end of said needle.

26. An apparatus according to claim 1, wherein said distal end of said stylet is adapted to cut said cytology sample.

27. An apparatus according to claim 26, wherein said distal end of said stylet is sharp.

28. An apparatus according to claim 26, wherein said distal end of said stylet is adapted to extend beyond the distal end of said needle.

29. An apparatus according to claim 1, wherein an outer surface of said stylet has a first diameter and an inner surface of said needle has a second diameter slightly larger than said first diameter, wherein said outer surface of said stylet is adapted to contact said inner surface of said needle to plug said inner lumen of said needle while said cytology sample is cut.

30. An apparatus according to claim 1, wherein said cytology collection device comprises a wire mesh device.

31. A medical apparatus for collecting a cytology sample from a mammalian body comprising:
an endoscope containing a transducer, said endoscope defining a working lumen;
a member having a proximal end, a distal end, and an inner lumen, wherein said member extends into the working lumen of said endoscope; and
a cytology collection device having a proximal end, and a distal end for cytology collection, wherein said cytology collection device is adapted to be inserted into said inner lumen of said member, with said distal end of said cytology collection device adapted to extend beyond the distal end of said member in order to collect said cytology sample, wherein said transducer is adapted to emit ultrasound waves to determine a position of said cytology collection device within said mammalian body using said ultrasound waves.

32. An apparatus according to claim 31, wherein said cytology collection device comprises an elongate member having a proximal end, a distal end, and a plurality of bristles.

33. An apparatus according to claim 32, wherein said elongate member comprises a dimpled wire and said transducer is adapted to emit ultrasound waves capable of reflecting off said dimpled wire.

34. An apparatus according to claim 32, wherein said transducer is adapted to emit ultrasound waves capable of reflecting off said plurality of bristles.

35. An apparatus according to claim 34, wherein said plurality of bristles is made of one or more of nylon, brass, stainless steel, metal, carbon, and polymer.

36. An apparatus according to claim 31, wherein said member is a needle.

37. An apparatus according to claim 31, wherein said distal end of said cytology collection device terminates in one of a cap, ball tip, cone tip, bevel tip, and loop.

38. An apparatus according to claim 37, wherein one of said cap, ball tip, cone tip, bevel tip, and loop is made of one or more of steel, metal, and solder.

39. An apparatus according to claim 32, wherein said elongate member comprises a wire and said wire is twisted around said plurality of bristles.

40. An apparatus according to claim 39, wherein said wire is made of one or more of steel, metal, and nitinol.

41. An apparatus according to claim 32, wherein the distal end of the elongate member is adapted to be retracted within the inner lumen of said member after collecting said cytology sample.

42. An apparatus according to claim 31, wherein said member is made of one or more of steel, and metal.

43. An apparatus according to claim 31, wherein said member is adapted to provide suction.

44. An apparatus according to claim 31, further comprising a handle which is adapted to provide axial movement of said cytology collection device and said member.

45. An apparatus according to claim 44, wherein said handle comprises:
an inner handle member having a proximal end and a distal end;
a first outer handle member slideably disposed on the inner handle member; and
an elongate sheath attached to the inner handle member and axially extending beyond the distal end of the inner handle member, the sheath defining a sheath lumen, wherein said member is attached to the first outer handle member and disposed in the sheath lumen.

46. An apparatus according to claim 44, wherein said handle comprises:
an inner handle member having proximal and distal ends and defining a handle lumen;
a first outer handle member slideably disposed on the proximal end of the inner handle member;
a second outer handle member slideably disposed on the distal end of the inner handle member;
an elongate sheath attached to the inner handle member and axially extending beyond the distal end of the inner handle member, the sheath defining a sheath lumen, wherein said member is attached to the first outer handle member, the member extending through the handle lumen and into the sheath lumen; and
a cap slideably disposed at the proximal end of the first outer handle member, said cap connected to the cytology collection device.

47. An apparatus according to claim 46, wherein said elongate sheath axially extends into the working lumen of the endoscope.

48. An apparatus according to claim 44, wherein said handle comprises one of a finger-loop handle, a pin-vice handle, and a half-finger loop handle.

49. An apparatus according to claim 31, wherein said cytology collection device comprises a balloon adapted to fit within said inner lumen of said member when deflated and adapted to inflate when outside of said inner lumen of said member in order to collect said cytology sample.

50. An apparatus according to claim 49, wherein an outer surface of said balloon is rough.

51. An apparatus according to claim 49, wherein said balloon is made of one or more of polymer, silicone, and polyethylene terephthalate.

52. An apparatus according to claim 31, further comprising a marker near said proximal end of said cytology collection device which is adapted to indicate when said distal end of said cytology collection device has been extended beyond the distal end of said member.

53. A method for collecting a cytology sample from a mammalian body comprising:
providing an apparatus comprising: a needle having a proximal end, a distal end, and an inner lumen extending from said proximal end to said distal end; a stylet having a proximal end and a distal end; and a cytology collection device having a proximal end and a distal end for cytology collection;
inserting said stylet into said inner lumen of said needle, wherein at least a portion of said stylet plugs said inner lumen of said needle;
cutting an area within said mammalian body;
withdrawing said stylet from said inner lumen of said needle;
inserting said cytology collection device into said inner lumen of said needle so that said distal end of said cytology collection device extends beyond the distal end of said needle;
collecting said cytology sample from said mammalian body using said cytology collection device; and
retracting said distal end of said cytology collection device into said inner lumen of said needle.

54. A method according to claim 53, wherein said cytology collection device comprises an elongate member having a proximal end, a distal end, and a plurality of bristles.

55. A method according to claim 53, wherein said distal end of said cytology collection device terminates in one of a cap, ball tip, cone tip, bevel tip, and loop.

56. A method according to claim 55, wherein one of said cap, ball tip, cone tip, bevel tip, and loop is made of one or more of steel, metal, and solder.

57. A method according to claim 54, wherein said elongate member comprises a wire and said wire is twisted around said plurality of bristles.

58. A method according to claim 57, wherein said wire is made of one or more of steel, metal, and nitinol.

59. A method according to claim 53, wherein said needle is made of one or more of steel, and metal.

60. A method according to claim 54, wherein said plurality of bristles is made of one or more of nylon, brass, stainless steel, metal, carbon, and polymer.

61. A method according to claim 53, further providing the step of providing suction through said needle.

62. A method according to claim 53, wherein the provided apparatus further comprises a handle which is adapted to provide at various points in time axial movement of said stylet, axial movement of said cytology collection device, and axial movement of said needle.

63. A method according to claim 62, wherein said handle comprises:
an inner handle member having a proximal end and a distal end;
a first outer handle member slideably disposed on the inner handle member; and
an elongate sheath attached to the inner handle member and axially extending beyond the distal end of the inner handle member, the sheath defining a sheath lumen, wherein said needle is attached to the first outer handle member and disposed in the sheath lumen.

64. A method according to claim 62, wherein said handle comprises:
an inner handle member having proximal and distal ends and defining a handle lumen;
a first outer handle member slideably disposed on the proximal end of the inner handle member;
a second outer handle member slideably disposed on the distal end of the inner handle member;
an elongate sheath attached to the inner handle member and axially extending beyond the distal end of the inner handle member, the sheath defining a sheath lumen, wherein said needle is attached to the first outer handle member, the needle extending through the handle lumen and into the sheath lumen; and
a cap slideably disposed at the proximal end of the first outer handle member, said cap connected to said stylet.

65. A method according to claim 64, wherein said cap is attached to the proximal end of said stylet, and the steps of inserting said stylet into the inner lumen of the needle, cutting an area within the body, and withdrawing the stylet from the inner lumen of the needle are all achieved by slideably moving said cap axially.

66. A method according to claim 62, wherein said handle comprises:
an inner handle member having proximal and distal ends and defining a handle lumen;
a first outer handle member slideably disposed on the proximal end of the inner handle member;
a second outer handle member slideably disposed on the distal end of the inner handle member;
an elongate sheath attached to the inner handle member and axially extending beyond the distal end of the inner handle member, the sheath defining a sheath lumen, wherein said needle is attached to the first outer handle member, the needle extending through the handle lumen and into the sheath lumen; and
a cap slideably disposed at the proximal end of the first outer handle member, said cap connected to said cytology collection device.

67. A method according to claim 66, wherein said cap is attached to the proximal end of said cytology collection device, and the steps of inserting said cytology collection device into the inner lumen of the needle, collecting the cytology sample from the body, and retracting the cytology collection device into the inner lumen of said needle are all achieved by slideably moving said cap axially.

68. A method according to claim 66, wherein the provided apparatus further comprises an endoscope defining a working lumen, wherein said elongate sheath axially extends into the working lumen of the endoscope.

69. A method according to claim 62, wherein said handle comprises one of a finger-loop handle, a pin-vice handle, and a half-finger loop handle.

70. A method according to claim 53, wherein the provided apparatus further comprises an endoscope containing a transducer adapted to emit ultrasound waves, and the method further comprises the additional step of determining a position of said cytology collection device within said mammalian body through ultrasound waves emitted by said transducer.

71. A method according to claim 70, wherein said cytology collection device comprises a dimpled wire having a proximal end, a distal end, and a plurality of bristles, wherein said transducer is adapted to emit ultrasound waves capable of reflecting off said dimpled wire.

72. A method according to claim 70, wherein said cytology collection device comprises an elongate member having a proximal end, a distal end, and a plurality of bristles, wherein said transducer is adapted to emit ultrasound waves capable of reflecting off said plurality of bristles.

73. A method according to claim 72, wherein said plurality of bristles is made of one or more of nylon, brass, stainless steel, metal, carbon, and polymer.

74. A method according to claim 53, wherein said stylet is a solid rod.

75. A method according to claim 53, wherein said stylet is made of one or more of steel, metal, and nitinol.

76. A method according to claim 53, wherein said cytology collection device comprises a balloon adapted to fit within said inner lumen of said needle when deflated and adapted to inflate when outside of said inner lumen of said needle in order to collect said cytology sample.

77. A method according to claim 76, wherein an outer surface of said balloon is rough.

78. A method according to claim 76, wherein said balloon is made of one or more of polymer, silicone, and polyethylene terephthalate.

79. A method according to claim 53, further comprising a marker near said proximal end of said cytology collection device which is adapted to indicate when said distal end of said cytology collection device has been extended beyond the distal end of said needle.

80. A method according to claim 53, wherein said distal end of said stylet is used to cut an area within said mammalian body.

81. A method according to claim 80, wherein said distal end of said stylet is sharp.

82. A method according to claim 80, wherein when said stylet is inserted into said inner lumen of said needle the distal end of said stylet is extended beyond the distal end of said needle.

83. A method according to claim 53, wherein an outer surface of said stylet has a first diameter and an inner surface of said needle has a second diameter slightly larger than said first diameter, wherein said outer surface of said stylet is adapted to contact said inner surface of said needle to plug said inner lumen of said needle while said area within said body is cut.

84. A method according to claim 53, wherein said cytology collection device comprises a wire mesh device.

85. A method for collecting a cytology sample from a mammalian body comprising:
providing an apparatus comprising: an endoscope, said endoscope defining a working lumen and containing a transducer; a member having a proximal end, a distal end, and an inner lumen, wherein said member extends into the working lumen of said endoscope; and a cytology collection device having a proximal end, and a distal end for cytology collection, wherein said cytology collection device extends into said inner lumen of said member;
inserting said working lumen of said endoscope into said mammalian body;
extending said distal end of said cytology collection device beyond the distal end of said member;
emitting ultrasound waves from said transducer of said endoscope;
reflecting said ultrasound waves off said cytology collection device;
receiving said reflected ultrasound waves using said transducer of said endoscope; and
determining a position of said cytology collection device within said mammalian body.

86. A method according to claim 85, wherein said cytology collection device comprises an elongate member having a proximal end, a distal end, and a plurality of bristles.

87. A method according to claim 85, wherein said distal end of said cytology collection device terminates in one of a cap, ball tip, cone tip, bevel tip, and loop.

88. A method according to claim 87, wherein one of said cap, ball tip, cone tip, bevel tip, and loop is made of one or more of steel, metal, and solder.

89. A method according to claim 86, wherein said elongate member comprises a wire, wherein said wire is twisted around said plurality of bristles.

90. A method according to claim 89, wherein said wire is made of one or more of steel, metal, and nitinol.

91. A method according to claim 85, wherein said member is made of one or more of steel, and metal.

92. A method according to claim 86, wherein said plurality of bristles is made of one or more of nylon, brass, stainless steel, metal, carbon, and polymer.

93. A method according to claim 85, further providing the step of providing suction through said member.

94. A method according to claim 85, wherein said member is a needle.

95. A method according to claim 85, wherein the provided apparatus further comprises a handle which is adapted to provide at various points in time axial movement of said member, and axial movement of said cytology collection device.

96. A method according to claim 95, wherein said handle comprises:
an inner handle member having a proximal end and a distal end;
a first outer handle member slideably disposed on the inner handle member; and
an elongate sheath attached to the inner handle member and axially extending beyond the distal end of the inner handle member, the sheath defining a sheath lumen, wherein said member is attached to the first outer handle member and disposed in the sheath lumen.

97. A method according to claim 95, wherein said handle comprises:
an inner handle member having proximal and distal ends and defining a handle lumen;
a first outer handle member slideably disposed on the proximal end of the inner handle member;
a second outer handle member slideably disposed on the distal end of the inner handle member;
an elongate sheath attached to the inner handle member and axially extending beyond the distal end of the inner handle member, the sheath defining a sheath lumen, wherein said member is attached to the first outer handle member, the member extending through the handle lumen and into the sheath lumen; and
a cap slideably disposed at the proximal end of the first outer handle member, said cap connected to said cytology collection device.

98. A method according to claim 97, wherein said cap is attached to the proximal end of said cytology collection device, and the step of extending said distal end of said cytology collection device beyond the distal end of the member is accomplished by slideably moving said cap axially.

99. A method according to claim 97, wherein said elongate sheath axially extends into the working lumen of the endoscope.

100. A method according to claim 95, wherein said handle comprises one of a finger-loop handle, a pin-vice handle, and a half-finger loop handle.

101. A method according to claim 85, wherein said cytology collection device comprises a dimpled wire having a proximal end, a distal end, and a plurality of bristles, wherein said transducer is adapted to emit ultrasound waves which are capable of reflecting off said dimpled wire.

102. A method according to claim 85, wherein said cytology collection device comprises an elongate member having a proximal end, a distal end, and a plurality of bristles, wherein said transducer is adapted to emit ultrasound waves which are capable of reflecting off said plurality of bristles.

103. A method according to claim 102, wherein said plurality of bristles is made of one or more of nylon, brass, stainless steel, metal, carbon, and polymer.

104. A method according to claim 85, wherein said cytology collection device comprises a balloon adapted to fit within said inner lumen of said member when deflated and adapted to inflate when outside of said inner lumen of said member in order to collect said cytology sample.

105. A method according to claim 104, wherein an outer surface of said balloon is rough.

106. A method according to claim 104, wherein said balloon is made of one or more of polymer, silicone, and polyethylene terephthalate.

107. A method according to claim 85, further comprising a marker near said proximal end of said cytology collection device which is adapted to indicate when said distal end of said cytology collection device has been extended beyond the distal end of said member.
